(19) United States
(12) Patent Application Publication
(10) Pub. No.: US 2013/0108626 A1
(43) Pub. Date: May 2, 2013

(54) BLOOD PLASMA BIOMARKERS FOR BEVACIZUMAB COMBINATION THERAPIES FOR TREATMENT OF PANCREATIC CANCER

(71) Applicant: F. Hoffmann-La Roche AG, Basel (CH)

(72) Inventors: Paul DELMAR, Basel (CH); Dorothee Foernzler, Lenzburg (CH); Friedemann Krause, Penzberg (DE); Stefan Scherer, South San Francisco, CA (US)

(73) Assignee: F. Hoffmann-La Roche AG, Basel (CH)

(21) Appl. No.: 13/728,333

(22) Filed: Dec. 27, 2012

Related U.S. Application Data
(63) Continuation of application No. PCT/EP2011/062226, filed on Jul. 18, 2011.

(30) Foreign Application Priority Data
Jul. 19, 2010 (EP) 10170004.5

(51) Int. Cl. G01N 33/68; A61K 39/395

(52) U.S. Cl. CPC G01N 33/6893; A61K 39/3955
USPC 424/133.1; 506/9; 436/501; 435/7.23; 435/7.92; 506/18

(57) ABSTRACT

The present invention provides methods for improving the treatment effect of a chemotherapy regimen of a patient suffering from pancreatic cancer, in particular metastatic pancreatic cancer by adding bevacizumab (Avastin®) to a chemotherapy regimen by determining the expression level, in particular the blood plasma expression level, of one or more of VEGFA, VEGFR2 and PLGF relative to control levels of patients diagnosed with pancreatic cancer, in particular metastatic pancreatic cancer. In particular, the present invention provides methods of improving the treatment effect, wherein the treatment effect is the overall survival and/or progression-free survival of the patient. The present invention further provides for methods for assessing the sensitivity or responsiveness of a patient to bevacizumab (Avastin®) in combination with a chemotherapy regimen, by determining the expression level, in particular the blood plasma expression level, of one or more of VEGFA, VEGFR2 and PLGF relative to control levels in patients diagnosed with pancreatic cancer, in particular metastatic pancreatic cancer.

### CROSS-REFERENCE TO RELATED APPLICATIONS

This application is a continuation of international application PCT/EP2011/062226, filed Jul. 18, 2011, which claims priority from European Patent Application 10170004.5, filed Jul. 19, 2010, the contents of which are incorporated herein by reference.

The present invention provides methods for improving the treatment effect of a chemotherapy regimen of a patient suffering from pancreatic cancer, in particular metastatic pancreatic cancer by adding bevacizumab (Avastin®) to a chemotherapy regimen by determining the expression level, in particular the blood plasma expression level, of one or more of VEGFA, VEGFR2 and PLGF relative to control levels of patients diagnosed with pancreatic cancer, in particular metastatic pancreatic cancer. In particular, the present invention provides methods of improving the treatment effect, wherein the treatment effect is the overall survival and/or progression-free survival of the patient. The present invention further provides for methods for assessing the sensitivity or responsiveness of a patient to bevacizumab (Avastin®) in combination with a chemotherapy regimen, by determining the expression level, in particular the blood plasma expression level, of one or more of VEGFA, VEGFR2 and PLGF relative to control levels in patients diagnosed with pancreatic cancer, in particular metastatic pancreatic cancer.

Accordingly, the present invention relates to the identification and selection of biomarkers of pancreatic cancer, in particular metastatic pancreatic cancer, that correlate with sensitivity or responsiveness to angiogenesis inhibitors, e.g., bevacizumab (Avastin®), in combination with chemotherapeutic regimens, such as gemcitabine-erlotinib (GE) therapy. In this respect, the invention relates to the use of (a) blood plasma specific expression profile(s) of one or more of VEGFA, VEGFR2 and PLGF relative to controls established in patients diagnosed with pancreatic cancer, in particular metastatic pancreatic cancer, to identify patients sensitive or responsive to the addition of angiogenesis inhibitors, e.g., bevacizumab (Avastin®), to standard chemotherapies. The invention further relates to methods for improving the treatment effect, in particular, the overall survival and/or progression-free survival of a patient suffering from pancreatic cancer, in particular metastatic pancreatic cancer, by the addition of angiogenesis inhibitors, e.g., bevacizumab (Avastin®), to standard chemotherapies, e.g., gemcitabine-erlotinib (GE) therapy, by determining (a) blood plasma specific expression level(s) of one or more of VEGFA, VEGFR2 and PLGF relative to control(s) in patients diagnosed with pancreatic cancer, in particular metastatic pancreatic cancer. The invention further provides for kits and compositions for identification of patients sensitive or responsive to angiogenesis inhibitors, in particular, bevacizumab (Avastin®), determined and defined in accordance with the methods of the present invention.

Angiogenesis is necessary for cancer development, regulating not only primary tumor size and growth but also impacting invasive and metastatic potential. Accordingly, the mechanisms mediating angiogenic processes have been investigated as potential targets for directed anti-cancer therapies. Early in the study of angiogenic modulators, the vascular endothelial growth factor (VEGF) signalling pathway was discovered to preferentially regulate angiogenic activity in multiple cancer types. This factor signals through VEGF Receptor 2 (VEGFR-2), the major VEGF signalling receptor that mediates angiogenesis. Multiple therapeutics have been developed to modulate this pathway at various points. These therapies include, among others, bevacizumab, sunitinib, sorafenib and vatalanib. Although the use of angiogenic inhibitors in the clinic has shown success, not all patients respond or fail to fully respond to angiogenesis inhibitor therapy. The mechanism(s) underlying such incomplete response is unknown. Therefore, there is an increasing need for the identification of patient subgroups sensitive or responsive to anti-angiogenic cancer therapy.

While a number of angiogenesis inhibitors are known, the most prominent angiogenesis inhibitor is bevacizumab (Avastin®). Bevacizumab is a recombinant humanized monoclonal IgG1 antibody that specifically binds and blocks the biological effects of VEGF (vascular endothelial growth factor). VEGF is a key driver of tumor angiogenesis—an essential process required for tumor growth and metastasis, i.e., the dissemination of the tumor to other parts of the body. Avastin® is approved in Europe for the treatment of the advanced stages of four common types of cancer: colorectal cancer, breast cancer, non-small cell lung cancer (NSCLC) and kidney cancer, which collectively cause over 2.5 million deaths each year. In the United States, Avastin® was the first anti-angiogenesis therapy approved by the FDA, and it is now approved for the treatment of five tumor types: colorectal cancer, non-small cell lung cancer, breast cancer, brain (glioblastoma) and kidney (renal cell carcinoma). Over half a million patients have been treated with Avastin so far, and a comprehensive clinical program with over 450 clinical trials is investigating the further use of Avastin in the treatment of multiple cancer types (including colorectal, breast, non-small cell lung, brain, gastric, ovarian and prostate) in different settings (e.g., advanced or early stage disease). Importantly, Avastin® has shown promise as a co-therapeutic, demonstrating efficacy when combined with a broad range of chemotherapies and other anti-cancer treatments. Phase-III studies have been published demonstrating the beneficial effects of combining bevacizumab with standard chemotherapeutic regimens (see, e.g., Saltz et al., 2008, J. Clin. Oncol., 26:2013-2019; Yang et al., 2008, Clin. Cancer Res., 14:5893-5899; Hurwitz et al., 2004, N. Engl. J. Med., 350:2335-2342). However, as in previous studies of angiogenic inhibitors, some of these phase-III studies have shown that a portion of patients experience incomplete response to the addition of bevacizumab (Avastin®) to their chemotherapeutic regimens.

Accordingly, there is a need for methods of determining those patients that respond to or are likely to respond to combination therapies comprising angiogenesis inhibitors, in particular, bevacizumab (Avastin®). Thus, the technical problem underlying the present invention is the provision of methods and means for the identification of (a) patient(s) suffering from or prone to suffer from pancreatic cancer, in particular metastatic pancreatic cancer, who may benefit from the addition of angiogenesis inhibitors, in particular, bevacizumab (Avastin®), to chemotherapeutic therapies, e.g., gemcitabine-erlotinib (GE) therapy.

The technical problem is solved by provision of the embodiments characterized in the claims.

The present invention, therefore, provides a method for improving the treatment effect of a chemotherapy regimen of a patient suffering from pancreatic cancer by adding bevacizumab to said chemotherapy regimen, said method comprising:
(a) determining the protein expression level of one or more of VEGFA, VEGFR2 and PLGF in a patient sample; and
(b) administering bevacizumab in combination with a chemotherapy regimen to the patient having an increased expression level of one or more of VEGFA, VEGFR2 and PLGF relative to control expression levels determined in patients diagnosed with pancreatic cancer.

The present invention relates to a method for improving the treatment effect of a chemotherapy regimen of a patient suffering from pancreatic cancer by adding bevacizumab to the chemotherapy regimen, said method comprising:
(a) obtaining a sample from said patient;
(b) determining the protein expression level of one or more of VEGFA, VEGFR2 and PLGF; and
(c) administering bevacizumab in combination with a chemotherapy regimen to the patient having an increased expression level of one or more of VEGFA, VEGFR2 and PLGF relative to control expression levels determined in patients diagnosed with pancreatic cancer.

The present invention relates to a method for improving the overall survival of a patient suffering from pancreatic cancer by adding bevacizumab to a chemotherapy regimen, said method comprising:
(a) determining the protein expression level of one or more of VEGFA, VEGFR2 and PLGF in a patient sample; and
(b) administering bevacizumab in combination with the chemotherapy regimen to the patient having an increased expression level of one or more of VEGFA, VEGFR2 and PLGF relative to control expression levels determined in patients diagnosed with pancreatic cancer.

The present invention relates to a method for improving the overall survival of a patient suffering from pancreatic cancer by adding bevacizumab to a chemotherapy regimen, said method comprising:
(a) obtaining a sample from said patient;
(b) determining the protein expression level of one or more of VEGFA, VEGFR2 and PLGF; and
(c) administering bevacizumab in combination with the chemotherapy regimen to the patient having an increased expression level of one or more of VEGFA, VEGFR2 and PLGF relative to control expression levels determined in patients diagnosed with pancreatic cancer.

The present invention relates to a method for improving the overall survival of a patient suffering from metastatic pancreatic cancer by adding bevacizumab to a chemotherapy regimen, said method comprising:
(a) determining the protein expression level of one or more of VEGFA, VEGFR2 and PLGF in a patient sample; and
(b) administering bevacizumab in combination with the chemotherapy regimen to the patient having an increased expression level of one or more of VEGFA, VEGFR2 and PLGF relative to control expression levels determined in patients diagnosed with metastatic pancreatic cancer,
   wherein the chemotherapy regimen comprises gemcitabine-erlotinib therapy.

The present invention relates to a method for improving the overall survival of a patient suffering from metastatic pancreatic cancer by adding bevacizumab to a chemotherapy regimen, said method comprising:
(a) obtaining a sample from said patient;
(b) determining the protein expression level of one or more of VEGFA, VEGFR2 and PLGF; and
(c) administering bevacizumab in combination with the chemotherapy regimen to the patient having an increased expression level of one or more of VEGFA, VEGFR2 and PLGF relative to control expression levels determined in patients diagnosed with metastatic pancreatic cancer,
   wherein the chemotherapy regimen comprises gemcitabine-erlotinib therapy.

The present invention relates to a method for improving the progression free survival of a patient suffering from pancreatic cancer by adding bevacizumab to a chemotherapy regimen, said method comprising:
(a) determining the protein expression level of one or more of VEGFA, VEGFR2 and PLGF in a patient sample; and
(b) administering bevacizumab in combination with the chemotherapy regimen to the patient having an increased expression level of one or more of VEGFA, VEGFR2 and PLGF relative to control expression levels determined in patients diagnosed with pancreatic cancer.

The present invention relates to a method for improving the progression free survival of a patient suffering from pancreatic cancer by adding bevacizumab to a chemotherapy regimen, said method comprising:
(a) obtaining a sample from said patient;
(b) determining the protein expression level of one or more of VEGFA, VEGFR2 and PLGF; and
(c) administering bevacizumab in combination with the chemotherapy regimen to the patient having an increased expression level of one or more of VEGFA, VEGFR2 and PLGF relative to control expression levels determined in patients diagnosed with pancreatic cancer.

The present invention relates to a method for improving the progression free survival of a patient suffering from metastatic pancreatic cancer by adding bevacizumab to a chemotherapy regimen, said method comprising:
(a) determining the protein expression level of one or more of VEGFA, VEGFR2 and PLGF in a patient sample; and
(b) administering bevacizumab in combination with the chemotherapy regimen to the patient having an increased expression level of one or more of VEGFA, VEGFR2 and PLGF relative to control expression levels determined in patients diagnosed with metastatic pancreatic cancer,
   wherein the chemotherapy regimen comprises gemcitabine-erlotinib therapy.

The present invention relates to a method for improving the progression free survival of a patient suffering from metastatic pancreatic cancer by adding bevacizumab to a chemotherapy regimen, said method comprising:
(a) obtaining a sample from said patient;
(b) determining the protein expression level of one or more of VEGFA, VEGFR2 and PLGF; and
(c) administering bevacizumab in combination with the chemotherapy regimen to the patient having an increased expression level of one or more of VEGFA, VEGFR2 and PLGF relative to control expression levels determined in patients diagnosed with metastatic pancreatic cancer,
   wherein the chemotherapy regimen comprises gemcitabine-erlotinib therapy.

The present invention relates to a method for improving the overall survival of a patient suffering from pancreatic cancer by adding bevacizumab to a chemotherapy regimen, said method comprising:
(a) determining the protein expression level of VEGFA or VEGFR2 in a patient sample; and
(b) administering bevacizumab in combination with the chemotherapy regimen to the patient having an increased expression level of VEGFA or VEGFR2 relative to control expression levels determined in patients diagnosed with pancreatic cancer.

The present invention relates to a method for improving the overall survival of a patient suffering from pancreatic cancer by adding bevacizumab to a chemotherapy regimen, said method comprising:
(a) obtaining a sample from said patient;
(b) determining the protein expression level of VEGFA or VEGFR2; and
(c) administering bevacizumab in combination with the chemotherapy regimen to the patient having an increased expression level VEGFA or VEGFR2 relative to control expression levels determined in patients diagnosed with pancreatic cancer.

The present invention relates to a method for improving the overall survival of a patient suffering from metastatic pancreatic cancer by adding bevacizumab to a chemotherapy regimen, said method comprising:
(a) determining the protein expression level of VEGFA or VEGFR2 in a patient sample; and
(b) administering bevacizumab in combination with the chemotherapy regimen to the patient having an increased expression level of VEGFA or VEGFR2 relative to control expression levels determined in patients diagnosed with metastatic pancreatic cancer,
   wherein the chemotherapy regimen comprises gemcitabine-erlotinib therapy.

The present invention relates to a method for improving the overall survival of a patient suffering from metastatic pancreatic cancer by adding bevacizumab to a chemotherapy regimen, said method comprising:
(a) obtaining a sample from said patient;
(b) determining the protein expression level of VEGFA or VEGFR2; and
(c) administering bevacizumab in combination with the chemotherapy regimen to the patient having an increased expression level of VEGFA or VEGFR2 relative to control expression levels determined in patients diagnosed with metastatic pancreatic cancer,
   wherein the chemotherapy regimen comprises gemcitabine-erlotinib therapy.

The present invention relates to a method for improving the progression free survival of a patient suffering from pancreatic cancer by adding bevacizumab to a chemotherapy regimen, said method comprising:
(a) determining the protein expression level of VEGFA or PLGF in a patient sample; and
(b) administering bevacizumab in combination with the chemotherapy regimen to the patient having an increased expression level of VEGFA or PLGF relative to control expression levels determined in patients diagnosed with pancreatic cancer.

The present invention relates to a method for improving the progression free survival of a patient suffering from pancreatic cancer by adding bevacizumab to a chemotherapy regimen, said method comprising:
(a) obtaining a sample from said patient;
(b) determining the protein expression level of VEGFA or PLGF; and
(c) administering bevacizumab in combination with the chemotherapy regimen to the patient having an increased expression level of VEGFA or PLGF relative to control expression levels determined in patients diagnosed with pancreatic cancer.

The present invention relates to a method for improving the progression free survival of a patient suffering from metastatic pancreatic cancer by adding bevacizumab to a chemotherapy regimen, said method comprising:
(a) determining the protein expression level of VEGFA or PLGF in a patient sample; and
(b) administering bevacizumab in combination with the chemotherapy regimen to the patient having an increased expression level of VEGFA or PLGF relative to control expression levels determined in patients diagnosed with metastatic pancreatic cancer,
   wherein the chemotherapy regimen comprises gemcitabine-erlotinib therapy.

The present invention relates to a method for improving the progression free survival of a patient suffering from metastatic pancreatic cancer by adding bevacizumab to a chemotherapy regimen, said method comprising:
(a) obtaining a sample from said patient;
(b) determining the protein expression level of VEGFA or PLGF; and
(c) administering bevacizumab in combination with the chemotherapy regimen to the patient having an increased expression level of VEGFA or PLGF relative to control expression levels determined in patients diagnosed with metastatic pancreatic cancer,
   wherein the chemotherapy regimen comprises gemcitabine-erlotinib therapy.

The invention provides a method for improving the overall survival of a patient suffering from pancreatic cancer by adding bevacizumab to a chemotherapy regimen, said method comprising:
(a) determining the protein expression level of VEGFA, VEGFR2 and PLGF in a patient sample; and
(b) administering bevacizumab in combination with a chemotherapy regimen to the patient having an increased combined expression level of VEGFA, VEGFR2 and PLGF relative to a control combined expression level determined in patients diagnosed with pancreatic cancer.

The pancreatic cancer may be metastatic pancreatic cancer.

Accordingly, the invention relates to a method for improving the overall survival of a patient suffering from pancreatic cancer by adding bevacizumab to a chemotherapy regimen, said method comprising:
(a) obtaining a sample from said patient;
(b) determining the protein expression level of VEGFA, VEGFR2 and PLGF; and
(c) administering bevacizumab in combination with a chemotherapy regimen to the patient having an increased combined expression level of VEGFA, VEGFR2 and PLGF relative to a control combined expression level determined in patients diagnosed with pancreatic cancer.

The pancreatic cancer may be metastatic pancreatic cancer.

The invention, therefore, relates to a method for improving the overall survival of a patient suffering from metastatic pancreatic cancer by adding bevacizumab to a chemotherapy regimen, said method comprising:
(a) determining the protein expression level of VEGFA, VEGFR2 and PLGF in a patient sample; and
(b) administering bevacizumab in combination the chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen comprises gemcitabine-erlotinib therapy.

The invention relates to a method for improving the overall survival of a patient suffering from metastatic pancreatic cancer by adding bevacizumab to a chemotherapy regimen, said method comprising:
(a) obtaining a sample from said patient;
(b) determining the protein expression level of VEGFA, VEGFR2 and PLGF; and
(c) administering bevacizumab in combination with the chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen comprises gemcitabine-erlotinib therapy.

The invention provides a method for improving the progression free survival of a patient suffering from pancreatic cancer by adding bevacizumab to a chemotherapy regimen, said method comprising:
(a) determining the protein expression level of VEGFA, VEGFR2 and PLGF in a patient sample; and
(b) administering bevacizumab in combination with a chemotherapy regimen to the patient having an increased combined expression level of VEGFA, VEGFR2 and PLGF relative to a control combined expression level determined in patients diagnosed with pancreatic cancer.

The pancreatic cancer may be metastatic pancreatic cancer.

Accordingly, the invention relates to a method for improving the progression free survival of a patient suffering from pancreatic cancer by adding bevacizumab to a chemotherapy regimen, said method comprising:
(a) obtaining a sample from said patient;
(b) determining the protein expression level of VEGFA, VEGFR2 and PLGF; and
(c) administering bevacizumab in combination with a chemotherapy regimen to the patient having an increased combined expression level of VEGFA, VEGFR2 and PLGF relative to a control combined expression level determined in patients diagnosed with pancreatic cancer.

The pancreatic cancer may be metastatic pancreatic cancer.

The invention, therefore, relates to a method for improving the progression free survival of a patient suffering from metastatic pancreatic cancer by adding bevacizumab to a chemotherapy regimen, said method comprising:
(a) determining the protein expression level of VEGFA, VEGFR2 and PLGF in a patient sample; and
(b) administering bevacizumab in combination the chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen comprises gemcitabine-erlotinib therapy.

The invention relates to a method for improving the progression free survival of a patient suffering from metastatic pancreatic cancer by adding bevacizumab to a chemotherapy regimen, said method comprising:
(a) obtaining a sample from said patient;
(b) determining the protein expression level of VEGFA, VEGFR2 and PLGF; and
(c) administering bevacizumab in combination with the chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen comprises gemcitabine-erlotinib therapy.

The invention provides a method for improving the overall survival of a patient suffering from metastatic pancreatic cancer by adding bevacizumab to a chemotherapy regimen, said method comprising:
(a) determining the protein expression level of VEGFA and VEGFR2 in a patient sample; and
(b) administering bevacizumab in combination with a chemotherapy regimen to the patient having an increased combined expression level of VEGFA and VEGFR2 relative to a control combined expression level determined in patients diagnosed with metastatic pancreatic cancer.

Accordingly, the invention relates to a method for improving the overall survival of a patient suffering from metastatic pancreatic cancer by adding bevacizumab to a chemotherapy regimen, said method comprising:
(a) obtaining a sample from said patient;
(b) determining the protein expression level of VEGFA and VEGFR2; and
(c) administering bevacizumab in combination with a chemotherapy regimen to the patient having an increased combined expression level of VEGFA and VEGFR2 relative to a control combined expression level determined in patients diagnosed with metastatic pancreatic cancer.

The invention, therefore, relates to a method for improving the overall survival of a patient suffering from metastatic pancreatic cancer by adding bevacizumab to a chemotherapy regimen, said method comprising:
(a) determining the protein expression level of VEGFA and VEGFR2 in a patient sample; and
(b) administering bevacizumab in combination the chemotherapy regimen to the patient having an increased combined expression level of VEGFA and VEGFR2 relative to a control combined expression level determined in patients diagnosed with metastatic pancreatic cancer,
   wherein the chemotherapy regimen comprises gemcitabine-erlotinib therapy.

The invention relates to a method for improving the overall survival of a patient suffering from metastatic pancreatic cancer by adding bevacizumab to a chemotherapy regimen, said method comprising:
(a) obtaining a sample from said patient;
(b) determining the protein expression level of VEGFA and VEGFR2; and
(c) administering bevacizumab in combination with the chemotherapy regimen to the patient having an increased combined expression level of VEGFA and VEGFR2 relative to a control combined expression level determined in patients diagnosed with metastatic pancreatic cancer,
   wherein the chemotherapy regimen comprises gemcitabine-erlotinib therapy.

The invention provides a method for improving the progression free survival of a patient suffering from metastatic pancreatic cancer by adding bevacizumab to a chemotherapy regimen, said method comprising:
(a) determining the protein expression level of VEGFA and VEGFR2 in a patient sample; and
(b) administering bevacizumab in combination with the chemotherapy regimen to the patient having an increased combined expression level of VEGFA and VEGFR2 relative to a control combined expression level determined in patients diagnosed with metastatic pancreatic cancer.

Accordingly, the invention relates to a method for improving the progression free survival of a patient suffering from metastatic pancreatic cancer by adding bevacizumab to a chemotherapy regimen, said method comprising:
(a) obtaining a sample from said patient;
(b) determining the protein expression level of VEGFA and VEGFR2; and
(c) administering bevacizumab in combination with the chemotherapy regimen to the patient having an increased combined expression level of VEGFA and VEGFR2 relative to a control combined expression level determined in patients diagnosed with metastatic pancreatic cancer.

The invention, therefore relates to a method for improving the progression free survival of a patient suffering from metastatic pancreatic cancer by adding bevacizumab to a chemotherapy regimen, said method comprising:
(a) determining the protein expression level of VEGFA and VEGFR2 in a patient sample; and
(b) administering bevacizumab in combination with the chemotherapy regimen to the patient having an increased combined expression level of VEGFA and VEGFR2 relative to a control combined expression level determined in patients diagnosed with metastatic pancreatic cancer,
   wherein the chemotherapy regimen comprises gemcitabine-erlotinib therapy.

The invention relates to a method for improving the progression free survival of a patient suffering from metastatic pancreatic cancer by adding bevacizumab to a chemotherapy regimen, said method comprising:
(a) obtaining a sample from said patient;
(b) determining the protein expression level of VEGFA and VEGFR2; and
(c) administering bevacizumab in combination with the chemotherapy regimen to the patient having an increased combined expression level of VEGFA and VEGFR2 relative to a control combined expression level determined in patients diagnosed with metastatic pancreatic cancer,
   wherein the chemotherapy regimen comprises gemcitabine-erlotinib therapy.

The present invention provides a method for improving the overall survival of a patient suffering from metastatic pancreatic cancer by adding bevacizumab to a chemotherapy regimen, said method comprising:
(a) determining the protein expression level of VEGFA and PLGF in a patient sample; and
(b) administering bevacizumab in combination with the chemotherapy regimen to the patient having an increased combined expression level of VEGFA and PLGF relative to a combined control expression level determined in patients diagnosed with metastatic pancreatic cancer.

Accordingly, the present invention relates to a method for improving the overall survival of a patient suffering from metastatic pancreatic cancer by adding bevacizumab to a chemotherapy regimen, said method comprising:
(a) obtaining a sample from said patient;
(b) determining the protein expression level of VEGFA and PLGF; and
(c) administering bevacizumab in combination with the chemotherapy regimen to the patient having an increased combined expression level of VEGFA and PLGF relative to a control combined expression level determined in patients diagnosed with metastatic pancreatic cancer.

The invention, therefore, relates to a method for improving the overall survival of a patient suffering from metastatic pancreatic cancer by adding bevacizumab to a chemotherapy regimen, said method comprising:
(a) determining the protein expression level of VEGFA and PLGF in a patient sample; and
(b) administering bevacizumab in combination with the chemotherapy regimen to the patient having an increased combined expression level of VEGFA and PLGF relative to a combined control expression level determined in patients diagnosed with metastatic pancreatic cancer,
   wherein the chemotherapy regimen comprises gemcitabine-erlotinib therapy.

Accordingly, the present invention relates to a method for improving the overall survival of a patient suffering from metastatic pancreatic cancer by adding bevacizumab to a chemotherapy regimen, said method comprising:
(a) obtaining a sample from said patient;
(b) determining the protein expression level of VEGFA and PLGF; and
(c) administering bevacizumab in combination with the chemotherapy regimen to the patient having an increased combined expression level of VEGFA and PLGF relative to a control combined expression level determined in patients diagnosed with metastatic pancreatic cancer,
   wherein the chemotherapy regimen comprises gemcitabine-erlotinib therapy.

The present invention provides a method for improving the progression-free survival of a patient suffering from metastatic pancreatic cancer by adding bevacizumab to a chemotherapy regimen, said method comprising:
(a) determining the protein expression level of VEGFA and PLGF in a patient sample; and
(b) administering bevacizumab in combination with the chemotherapy regimen to the patient having an increased combined expression level of VEGFA and PLGF relative to a combined control expression level determined in patients diagnosed with metastatic pancreatic cancer.

Accordingly, the present invention relates to a method for improving the progression free survival of a patient suffering from metastatic pancreatic cancer by adding bevacizumab to a chemotherapy regimen, said method comprising:
(a) obtaining a sample from said patient;
(b) determining the protein expression level of VEGFA and PLGF; and
(c) administering bevacizumab in combination with the chemotherapy regimen to the patient having an increased combined expression level of VEGFA and PLGF relative to a control combined expression level determined in patients diagnosed with metastatic pancreatic cancer.

The invention, therefore, relates to a method for improving the progression free survival of a patient suffering from metastatic pancreatic cancer by adding bevacizumab to a chemotherapy regimen, said method comprising:
(a) determining the protein expression level of VEGFA and PLGF in a patient sample; and
(b) administering bevacizumab in combination with the chemotherapy regimen to the patient having an increased combined expression level of VEGFA and PLGF relative to a combined control expression level determined in patients diagnosed with metastatic pancreatic cancer,
   wherein the chemotherapy regimen comprises gemcitabine-erlotinib therapy.

Accordingly, the present invention relates to a method for improving the progression free survival of a patient suffering from metastatic pancreatic cancer by adding bevacizumab to a chemotherapy regimen, said method comprising:
(a) obtaining a sample from said patient;
(b) determining the protein expression level of VEGFA and PLGF; and
(c) administering bevacizumab in combination with the chemotherapy regimen to the patient having an increased combined expression level of VEGFA and PLGF relative to a control combined expression level determined in patients diagnosed with metastatic pancreatic cancer,
   wherein the chemotherapy regimen comprises gemcitabine-erlotinib therapy.

The present invention provides an in vitro method for the identification of a patient responsive to or sensitive to the addition of bevacizumab treatment to a chemotherapy regimen, said method comprising determining the protein expression level of one or more of VEGFA, VEGFR2 and PLGF in a sample from a patient suffering from, suspected to suffer from or being prone to suffer from pancreatic cancer, in particular metastatic pancreatic cancer, whereby an increased expression level of one or more of VEGFA, VEGFR2 and PLGF relative to control expression levels determined in patients suffering from pancreatic cancer, in particular metastatic pancreatic cancer, is indicative of a sensitivity of the patient to the addition of bevacizumab to said chemotherapy regimen. The chemotherapy regimen may comprise gemcitabine-erlotinib therapy.

Accordingly, the present invention relates to an in vitro method for the identification of a patient responsive to or sensitive to the addition of bevacizumab treatment to a chemotherapy regimen, said method comprising:
(a) obtaining a sample from a patient suffering from, suspected to suffer from or being prone to suffer from pancreatic cancer, in particular metastatic pancreatic cancer; and
(b) determining the protein expression level of one or more of VEGFA, VEGFR2 and PLGF;
   whereby an increased expression level of one or more of VEGFA, VEGFR2 and PLGF relative to control expression levels determined in patients suffering from pancreatic cancer, in particular metastatic pancreatic cancer, is indicative of a sensitivity of the patient to the addition of bevacizumab to said chemotherapy regimen. The chemotherapy regimen may comprise gemcitabine-erlotinib therapy.

The present invention provides an in vitro method for the identification of a patient that is responsive to or sensitive to the addition of bevacizumab treatment to a chemotherapy regimen, said method comprising determining the protein expression level of one or more of VEGFA, VEGFR2 and PLGF in a sample from a patient suffering from, suspected to suffer from or being prone to suffer from metastatic pancreatic cancer, whereby an increased combined expression increased level of VEGFA and VEGFR2 or VEGFA and PLGF or VEGFA, VEGFR2 and PLGF relative to a control combined expression level determined in patients suffering from metastatic pancreatic cancer is indicative of a sensitivity of the patient to the addition of bevacizumab to said chemotherapy regimen. The chemotherapy regimen may comprise gemcitabine-erlotinib therapy.

Accordingly, the present invention solves the identified technical problem in that it was surprisingly shown that the blood plasma specific expression levels of one or more of VEGFA, VEGFR2 and PLGF in a given patient, relative to control levels determined in patients diagnosed with pancreatic cancer, in particular, metastatic pancreatic, correlate with treatment effect in those patients administered an angiogenesis inhibitor in combination with a chemotherapy regimen. Specifically, variations in the protein expression levels of VEGFA, VEGFR2 and/or PLGF were surprisingly identified as markers/predictors for the improved overall survival and/or progression-free survival of metastatic pancreatic cancer patients in response to the addition of bevacizumab (Avastin®) to the chemotherapy regimen of gemcitabine-erlotinib. Patients exhibiting a response or sensitivity to the addition of bevacizumab (Avastin®) to chemotherapy regimens were identified to have an increased protein expression level of one or more VEGFA, VEGFR2 and PLGF relative to control expression levels established in samples obtained from patients diagnosed with pancreatic cancer, in particular, metastatic pancreatic cancer. The terms “marker” and “predictor” can be used interchangeably and refer to the expression levels of one or more of VEGFA, VEGFR2 and PLGF as described herein. The invention also encompasses the use of the terms “marker” and “predictor” to refer to a combination of any two or more of the blood plasma expression levels of VEGFA, VEGFR2 and PLGF.

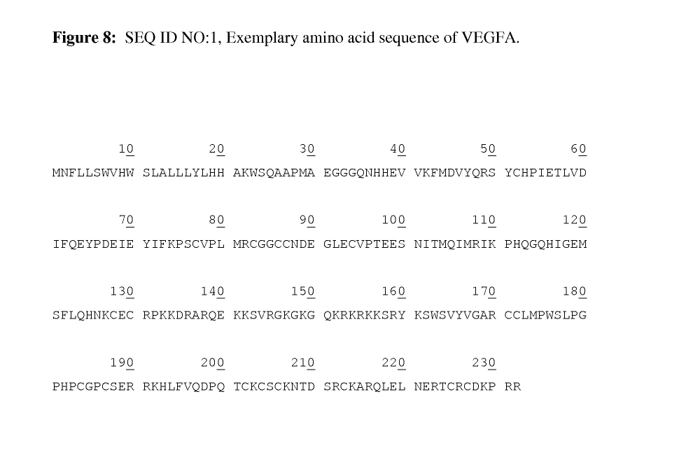

In the context of the present invention, “VEGFA” refers to vascular endothelial growth factor protein A, exemplified by SEQ ID NO:1, shown in FIG. 8 (Swiss Prot Accession Number P15692, Gene ID (NCBI): 7422). The term “VEGFA” encompasses the protein having the amino acid sequence of SEQ ID NO:1 as well as homologues and isoforms thereof. The term “VEGFA” also encompasses the known isoforms, e.g., splice isoforms, of VEGFA, e.g., VEGF₁₁₁, VEGF₁₂₁, VEGF₁₄₅, VEGF₁₆₅, VEGF₁₈₉ and VEGF₂₀₆, as well as variants, homologues and isoforms thereof, including the 110-amino acid human vascular endothelial cell growth factor generated by plasmin cleavage of VEGF₁₆₅ as described in Ferrara Mol. Biol. Cell 21:687 (2010) and Leung et al. Science 246:1306 (1989), and Houck et al. Mol. Endocrin. 5:1806 (1991). In a particular embodiment of the present invention, “VEGFA” refers to VEGF₁₂₁ and/or VEGF₁₁₀. In a particular embodiment of the present invention, “VEGFA” refers to VEGF₁₁₁. In the context of the invention, the term “VEGFA” also encompasses proteins having at least 85%, at least 90% or at least 95% homology to the amino acid sequence of SEQ ID NO:1, or to the amino acid sequences of the variants and/or homologues thereof, as well as fragments of the sequences, provided that the variant proteins (including isoforms), homologous proteins and/or fragments are recognized by one or more VEGFA specific antibodies, such as antibody clone 3C5 and 26503, which are available from Bender RELIATech and R&D Systems, respectively and A4.6.1 as described in Kim et al., Growth Factors 7(1): 53-64 (1992). In the context of the invention, the term “isoform” of VEGF or VEGF-A refers to both splice isoforms and forms generated by enzymatic cleavage (e.g., plasmin).

In one embodiment, “VEGFA” refers to unmodified VEGF. In the context of the present invention “unmodified” VEGF relates to the unmodified amino acid sequence of VEGF, its isoforms and its cleavage products. Unmodified VEGF can e.g. be produced synthetically or preferably recombinantly in prokaryotic expression systems, e.g. in E. coli. Unmodified VEGF does e.g. not carry a posttranslational modification, like a glycosylation. In the context of the invention, the term “unmodified VEGF-A” also encompasses variants and/or homologues thereof, as well as fragments of VEGF-A, provided that the variant proteins (including isoforms), homologous proteins and/or fragments are recognized by an unmodified VEGF-A specific antibodies, such as antibody clone 3C5, which is available from RELIATech GmbH, Wolfenbüttel, Germany.

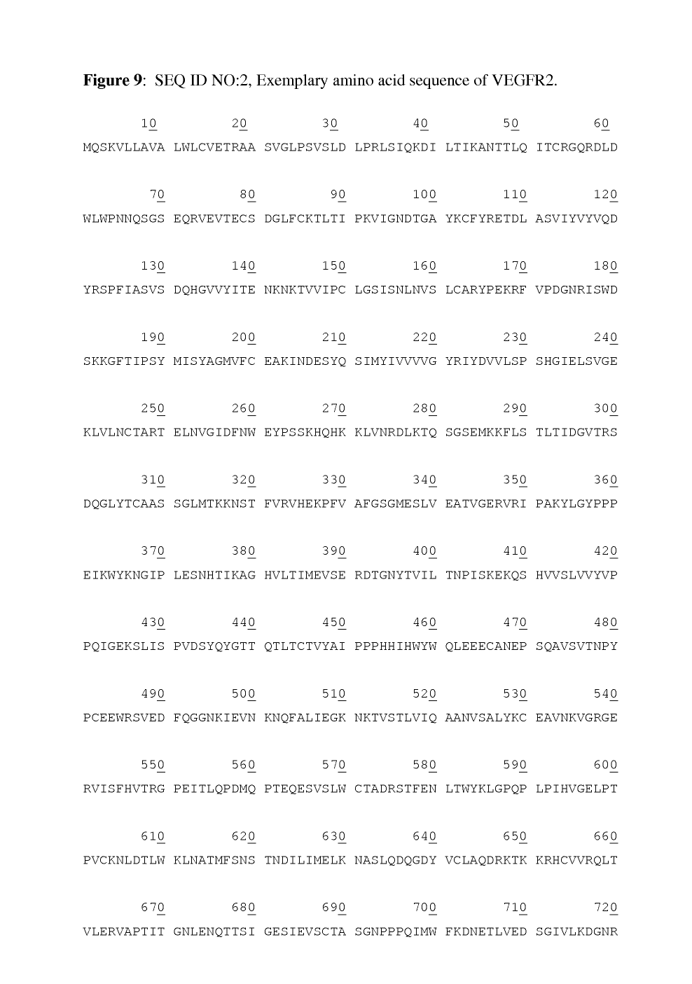

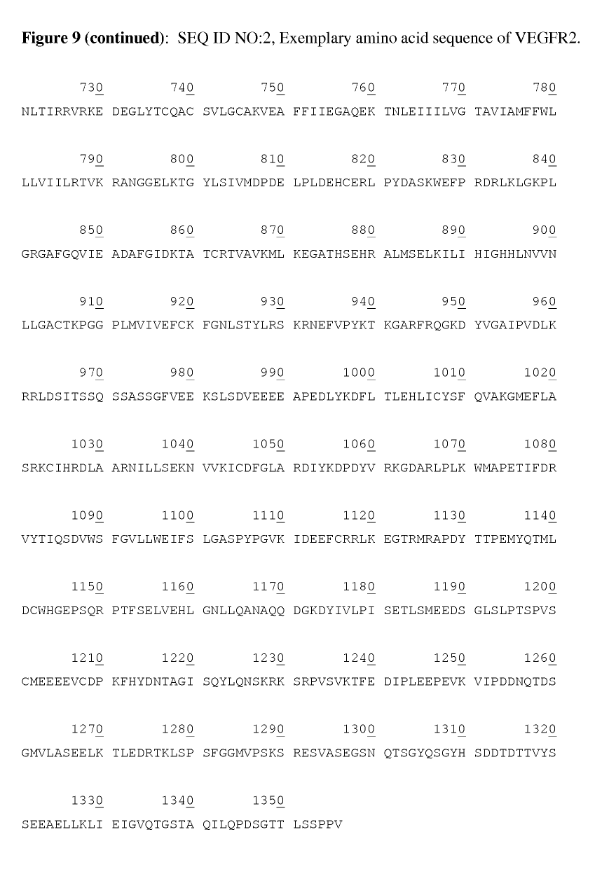

In the context of the present invention, “VEGFR2” refers to vascular endothelial growth factor receptor 2, exemplified by SEQ ID NO:2, shown in FIG. 9 (Swiss Prot Accession Number P35968, Gene ID (NCBI): 3791). The term “VEGFR2” encompasses the protein having the amino acid sequence of SEQ ID NO:2 as well as homologues and isoforms thereof. In the context of the invention, the term “VEGFR2” also encompasses proteins having at least 85%, at least 90% or at least 95% homology to the amino acid sequence of SEQ ID NO:2, or to the amino acid sequences of the variants and/or homologues thereof, as well as fragments of the sequences, provided that the variant proteins (including isoforms), homologous proteins and/or fragments are recognized by one or more VEGFR2 specific antibodies, such as antibody clone 89115 and 89109, which are available from R&D Systems.

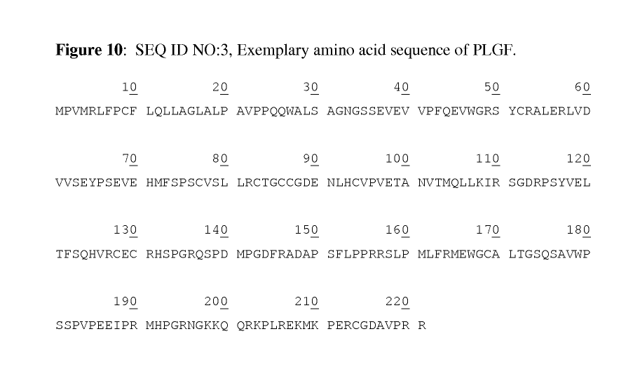

In the context of the present invention, “PLGF” refers to placental growth factor exemplified by SEQ ID NO:3, shown in FIG. 10 (Swiss Prot Accession Number P49763, Gene ID (NCBI): 5228). The term “PLGF” encompasses the protein having the amino acid sequence of SEQ ID NO:3 as well as homologues and isoforms thereof. In the context of the invention, the term “PLGF” also encompasses proteins having at least 85%, at least 90% or at least 95% homology to the amino acid sequence of SEQ ID NO:3, or to the amino acid sequences of the variants and/or homologues thereof, as well as fragments of the sequences, provided that the variant proteins (including isoforms), homologous proteins and/or fragments are recognized by one or more PLGF specific antibodies, such as antibody clone 2D6D5 and 6A11D2, which are available from Roche Diagnostics GmbH.

Accordingly, the present invention encompasses the determination of expression levels of proteins including, but not limited to, the amino acid sequences as described herein. In this context the invention encompasses the detection of homologues, variants and isoforms of one or more of VEGFA, VEGFR2 and PLGF; said isoforms or variants may, inter alia, comprise allelic variants or splice variants. Also envisaged is the detection of proteins that are homologous to one or more of VEGFA, VEGFR2 and PLGF as herein described, or a fragment thereof, e.g., having at least 60%, 70%, 80%, 90%, 95%, 96%, 97%, 98% or 99% sequence identity to the amino acid sequence of SEQ ID NO:1, SEQ ID NO:2 or SEQ ID NO:3 or a fragment thereof. Alternatively or additionally, the present invention encompasses detection of the expression levels of proteins encoded by nucleic acid sequences, or fragments thereof, that are at least at least 60%, 70%, 80%, 90%, 95%, 96%, 97%, 98% or 99% identical to a nucleic acid sequence encoding SEQ ID NO:1, SEQ ID NO:2 or SEQ ID NO:3 or a fragment, variant or isoform thereof. In this context, the term “variant” means that the VEGFA, VEGFR2 and/or PLGF amino acid sequence, or the nucleic acid sequence encoding said amino acid sequence, differs from the distinct sequences identified by SEQ ID NOs:1, SEQ ID NO:2 or SEQ ID NO:3 and/or available under the above-identified Swiss Prot Accession numbers, by mutations, e.g., deletion, additions, substitutions, inversions etc. In addition, the term “homologue” references molecules having at least 60%, more preferably at least 80% and most preferably at least 90% sequence identity to one or more of the polypeptides as shown in SEQ ID NOs:1, SEQ ID NO:2 or SEQ ID NO:3, or (a) fragment(s) thereof.

In order to determine whether an amino acid or nucleic acid sequence has a certain degree of identity to an amino acid or nucleic acid sequence as herein described, the skilled person can use means and methods well known in the art, e.g. alignments, either manually or by using computer programs known in the art or described herein.

In accordance with the present invention, the term “identical” or “percent identity” in the context of two or more or amino acid or nucleic acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the amino acid sequences of, e.g., SEQ ID NO:1, SEQ ID NO:2 or SEQ ID NO:3), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST (Basic Local Alignment Search Tool) and BLAST 2.0 algorithms (Altschul, 1997, Nucl. Acids Res. 25:3389-3402; Altschul, 1993 J. Mol. Evol. 36:290-300; Altschul, 1990, J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

BLAST algorithms, as discussed above, produce alignments of both amino and nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST may be used to search for identical or related molecules in protein or nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identit y \times \% maximum BLAST score}{100}$$

and takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson, 1994, Nucl. Acids Res. 2:4673-4680) or FASTDB (Brutlag, 1990, Comp. App. Biosci. 6:237-245), as is known in the art.

In the context of the herein described invention, the expression levels, in particular protein expression levels, of VEGFA, VEGFR2 and/or PLGF, may be considered separately, as individual markers, or in groups of two or more, as an expression profile or marker panel. In the context of the herein described invention an expression profile or marker panel wherein the expression profiles of two or more markers may be considered together may also be referred to as a combined expression level. For example, the expression levels of two or more markers may be added together and compared to a similarly determined control combined expression level. Therefore, the methods of the invention encompass determination of an expression profile, including a combined expression level, based on the expression level of one or more of the markers.

In the context of the herein described invention, and in accordance with the appended illustrative example, for consideration of VEGFA, VEGFR2 or PLGF separately, the following values were used as the corresponding high or low expression value of the marker: High VEGFA (≧152.9 pg/ml), Low VEGFA (<152.9 pg/ml), High VEGFR2 (≧9.9 ng/ml), Low VEGFR2 (<9.9 ng/ml). These levels were determined by the median of available samples as per pre-determined statistical analysis plan. Additionally, optimized levels constituting the cut-off value between high and low expression of a particular marker may be determined by varying the cut-off until the subset of patients above and below the cut-off satisfy a relevant statistical optimality criterion. For example, optimal cut-point may be chosen to maximize the differences in treatment Hazard Ratio between the subset above and below, or to maximize treatment effect in one sub-group, or any other relevant statistical criterion. In accordance with the herein described invention, and in accordance with the appended illustrative example, the optimized expression values for PLGF considered separately were High PLGF (≧36.5 pg/ml), and Low PLGF (<36.5 pg/ml). This level was determined as 42nd percentile of available data. This level was determined in order to increase the statistical difference in treatment effect between high and low level. The skilled person will, however, understand that the expression level of the particular marker and, therefore, what constitutes a high or low expression level may vary by patient and by patient population. Accordingly, the skilled person will understand that when using detections methods other than those described in the appended illustrative example and studying patients and patient populations other than those described in the appended illustrative example, what the skilled person considers a high and/or low expression level for a particular biomarker may vary from the values herein described. Given the methods herein described, the skilled person can determine what constitutes a high and/or low level of expression of a particular biomarker.

As the skilled person will appreciate there are many ways to use the measurements of two or more markers in order to improve the diagnostic question under investigation. In a quite simple, but nonetheless often effective approach, a positive result is assumed if a sample is positive for at least one of the markers investigated.

However, a combination of markers may also be evaluated. The values measured for markers of a marker panel (or a combined expression level), e.g. for VEGFA and VEGFR2 or VEGFA and PLGF or VEGFA, VEGFR2 and PLGF, may be mathematically combined and the combined value may be correlated to the underlying diagnostic question. Marker values may be combined by any appropriate state of the art mathematical method. Well-known mathematical methods for correlating a marker combination to a disease or to a treatment effect employ methods like, discriminant analysis (DA) (i.e. linear-, quadratic-, regularized-DA), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting/Bagging Methods), Generalized Linear Models (i.e. Logistic Regression), Principal Components based Methods (i.e. SIMCA), Generalized Additive Models, Fuzzy Logic based Methods, Neural Networks and Genetic Algorithms based Methods. The skilled artisan will have no problem selecting an appropriate method to evaluate a marker combination of the present invention. The method used in correlating marker combinations in accordance with the invention herein disclosed with, for example improved overall survival, progression free survival, responsiveness or sensitivity to addition of bevacizumab to chemotherapeutic agents/chemotherapy regimen and/or the prediction of a response to or sensitivity to bevacizumab (in addition to one or more chemotherapeutic agents/chemotherapy regimen) is selected from DA (i.e. Linear-, Quadratic-, Regularized Discriminant Analysis), Kernel Methods (i.e. SVM), Nonparametric Methods (i.e. k-Nearest-Neighbor Classifiers), PLS (Partial Least Squares), Tree-Based Methods (i.e. Logic Regression, CART, Random Forest Methods, Boosting Methods), or Generalized Linear Models (i.e. Logistic Regression). Details relating to these statistical methods are found in the following references: Ruczinski, I., et al, J. of Computational and Graphical Statistics, 12 (2003) 475-511; Friedman, J. H., J. of the American Statistical Association 84 (1989) 165-175; Hastie, Trevor, Tibshirani, Robert, Friedman, Jerome, The Elements of Statistical Learning, Springer Series in Statistics, 2001; Breiman, L., Friedman, J. H., Olshen, R. A., Stone, C. J. (1984) Classification and regression trees, California: Wadsworth; Breiman, L., Random Forests, Machine Learning, 45 (2001) 5-32; Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

Accordingly, the invention herein disclosed relates to the use of an optimized multivariate cut-off for the underlying combination of biological markers and to discriminate state A from state B, e.g. patients responsive to or sensitive to the addition of bevacizumab to a chemotherapy regimen from patients that are poor responders to the addition of bevacizumab therapy to a chemotherapy regimen. In this type of analysis the markers are no longer independent but form a marker panel or a combined expression level.

The present invention, therefore, relates to method for improving the treatment effect of a chemotherapy regimen of patients suffering from pancreatic cancer, in particular metastatic pancreatic cancer, by adding bevacizumab to a chemotherapy regimen by determining the expression levels two or more of VEGFA, PLGF and VEGFR2, by adding these expression levels such that each expression level is multiplied with a weight function (or weighting factor). Surprisingly, the result (“value”, result of the mathematical operation, or combined expression level) correlates with treatment effect in patients administered bevacizumab in combination chemotherapy regimens such that values above a pre-specified (multivariate) cut-off are indicative of better treatment effect for the patient and values below this cut-off are indicative of poorer treatment effect.

The present invention, accordingly, relates to a method for improving the treatment effect of a chemotherapy regimen of patients suffering from cancer, in particular metastatic pancreatic cancer, by adding bevacizumab to a chemotherapy regimen by determining the expression levels of VEGFA and VEGFR2, and by adding these expression levels such that each expression level is multiplied with a weight function (or weighting factor). Surprisingly, the result (“value”, result of the mathematical operation, or combined expression level) correlates with treatment effect in patients administered bevacizumab in combination chemotherapy regimens such that values above a pre-specified (multivariate) cut-off are indicative of better treatment effect for the patient and values below this cut-off are indicative of poorer treatment effect.

The present invention also relates to a method for improving the treatment effect of a chemotherapy regimen of patients suffering from cancer, in particular metastatic pancreatic cancer, by adding bevacizumab to a chemotherapy regimen by determining the expression levels of VEGFA and PLGF, and by adding these expression levels such that each expression level is multiplied with a weight function (or weighting factor). Surprisingly, the result (“value”, result of the mathematical operation, or combined expression level) correlates with treatment effect in patients administered bevacizumab in combination chemotherapy regimens such that values above a pre-specified (multivariate) cut-off are indicative of better treatment effect for the patient and values below this cut-off are indicative of poorer treatment effect.

The present invention relates to a method for improving the treatment effect of a chemotherapy regimen of patients suffering from cancer, in particular metastatic pancreatic cancer, by adding bevacizumab to a chemotherapy regimen by determining the expression levels of VEGFA, VEGFR2 and PLGF, and by adding these expression levels such that each expression level is multiplied with a weight function (or weighting factor). Surprisingly, the result (“value”, result of the mathematical operation, or combined expression level) correlates with treatment effect in patients administered bevacizumab in combination chemotherapy regimens such that values above a pre-specified (multivariate) cut-off are indicative of better treatment effect for the patient and values below this cut-off are indicative of poorer treatment effect.

For example, as shown in the appended illustrative example, the following equations can be used for assessing the combined expression level of VEGFA and VEGFR2 or VEGFA and PLGF when the treatment effect is overall survival in patients suffering from metastatic pancreatic cancer.

norm(VEGFA)+1.3*norm(VEGFR2). Cut-point=median or 0 Formula 1

EGFA+3.3*VEGFR2. Cut-point=median or 0 Equivalent formula

and

0.25*norm(VEGFA)+0.21*norm(PLGF), cut-point=median or 0 Formula 2

0.19*VEGFA+0.67*PLGF, cut-point=median or 4.8 Equivalent formula

Where we use log 2 transformation and

$${x}_{i} \to norm ( {x}_{i} ) = \frac{log 2 ( {x}_{i} ) - median ( log 2 ( x ) )}{mad ( log 2 ( x ) )}$$

Accordingly, in the context of the herein described invention, and in accordance with the appended illustrative example, a high combined expression level of VEGFA and VEGFR2 is (Formula 1≧−0.1) and a low combined expression of VEGFA and VEGFR2 is (Formula 1<−0.1), with regard to overall survival. In the context of the herein described invention, and in accordance with the appended illustrative example, a high combined expression level of VEGFA and PLGF is (Formula 2≧−0.042) and a low combined expression of VEGFA and PLGF is (Formula 2<−0.042), with regard to overall survival. The skilled person will, however, understand that the expression levels measured for markers of a marker panel (or a combined expression level), e.g. for VEGFA and VEGFR2 or VEGFA and PLGF, may be mathematically combined and the combined expression level may be correlated to the underlying diagnostic question in more than one way. Accordingly, marker levels may be combined by any appropriate state of the art mathematical method.

As is also shown in the appended illustrative example, the following equations can be used for assessing the combined expression level of VEGFA and VEGFR2 or VEGFA and PLGF when the treatment effect is progression free survival in patients suffering from metastatic pancreatic cancer.

norm(VEGFA)+1.3*norm(VEGFR2). Cut-point=median or 0 Formula 1

VEGFA+3.3*VEGFR2. Cut-point=median or 0 Equivalent formula

and

0.25*norm(VEGFA)+0.21*norm(PLGF), cut-point=median or 0 Formula 2

0.19*VEGFA+0.67*PLGF, cut-point=median or 4.8 Equivalent formula

Where we use log 2 transformation and

$${x}_{i} \to norm ( {x}_{i} ) = \frac{log 2 ( {x}_{i} ) - median ( log 2 ( x ) )}{mad ( log 2 ( x ) )}$$

Accordingly, in the context of the herein described invention, and in accordance with the appended illustrative example, a high combined expression level of VEGFA and VEGFR2 is (Formula 1≧−0.1) and a low combined expression of VEGFA and VEGFR2 is (Formula 1<−0.1), with regard to progression free survival. In the context of the herein described invention, and in accordance with the appended illustrative example, a high combined expression level of VEGFA and PLGF is (Formula 2≧−0.042) and a low combined expression of VEGFA and PLGF is (Formula 2<−0.042), with regard to progression free survival. The skilled person will, however, understand that the expression levels measured for markers of a marker panel (or a combined expression level), e.g. for VEGFA and VEGFR2 or VEGFA and PLGF, may be mathematically combined and the combined expression level may be correlated to the underlying diagnostic question in more than one way. Accordingly, marker levels may be combined by any appropriate state of the art mathematical method.

For example, as shown in the appended illustrative example, the following equation can be used for assessing the combined expression level of VEGFA, VEGFR2 and PLGF when the treatment effect is overall survival or progression free survival in patients suffering from metastatic pancreatic cancer.

0.0127*ln(PLGF+1)+0.144*ln(VEGFR2+1)+0.0949*ln(VEGFA+1) Formula 3

Where ln=log basis e

Accordingly, in the context of the herein described invention, and in accordance with the appended illustrative example, a high combined expression level of VEGFA, VEGFR2 and PLGF is (Formula 3≧0.837) and a low combined expression of VEGFA, VEGFR2 and PLGF is (Formula 3 <0.837), with regard to overall survival. In the context of the herein described invention, and in accordance with the appended illustrative example, a high combined expression level of VEGFA, VEGFR2 and PLGF is (Formula 3≧0.837) and a low combined expression of VEGFA, VEGFR2 and PLGF is (Formula 3<0.837), with regard to progression free survival. The skilled person will, however, understand that the expression levels measured for markers of a marker panel (or a combined expression level), e.g. for VEGFA, VEGFR2 and PLGF, may be mathematically combined and the combined expression level may be correlated to the underlying diagnostic question in more than one way. Accordingly, marker levels may be combined by any appropriate state of the art mathematical method.

The expression level of one or more of the markers VEGFA, VEGFR2 and PLGF may be assessed by any method known in the art suitable for determination of specific protein levels in a patient sample and is preferably determined by an immunoassay method, such as ELISA, employing antibodies specific for one or more of VEGFA, VEGFR2 and PLGF. Such methods are well known and routinely implemented in the art and corresponding commercial antibodies and/or kits are readily available. For example, commercially available antibodies/test kits for VEGFA, VEGFR2 and PLGF can be obtained from Bender RELIATech and R&D Systems as clone 3C5 and 26503, from R&D systems as clone 89115 and 89109 and from Roche Diagnostics GmbH as clone 2D6D5 and 6A11D2, respectively. Preferably, the expression levels of the marker/indicator proteins of the invention are assessed using the reagents and/or protocol recommendations of the antibody or kit manufacturer. The skilled person will also be aware of further means for determining the expression level of one or more of VEGFA, VEGFR2 and PLGF by immunoassay methods. Therefore, the expression level of one or more of the markers/indicators of the invention can be routinely and reproducibly determined by a person skilled in the art without undue burden. However, to ensure accurate and reproducible results, the invention also encompasses the testing of patient samples in a specialized laboratory that can ensure the validation of testing procedures.

VEGF₁₂₁ and VEGF₁₁₀ protein can be detected using any method known in the art. For example, tissue or cell samples from mammals can be conveniently assayed for, e.g., proteins using Westerns, ELISAs, etc. Many references are available to provide guidance in applying the above techniques (Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hunell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987)).

If reference is made to the detection or level of VEGF₁₂₁ and VEGF₁₁₀ this means that the sum of both molecules is measured, e.g., using an assay that detects both VEGF₁₂₁ and VEGF₁₁₀. Assays that detect both molecules VEGF₁₂₁ and VEGF₁₁₀ include, e.g., assays that have a sensitivity for the corresponding other form, (i.e. for VEGF₁₂₁ if VEGF₁₁₀ is better recognized, or for VEGF₁₁₀ if VEGF₁₂₁ is better recognized, respectively) of at least 25%. In certain embodiments, in the assays have sensitivity to the corresponding other form of at least 50%, 75%, 80%, 85%, 90% or above. In one embodiment both VEGF₁₂₁ and VEGF₁₁₀ are measured with essentially the same sensitivity.

As to detection of VEGF₁₂₁ and VEGF₁₁₀ protein, various assays are available. For example, the sample may be contacted with an antibody or an antibody combination (e.g. in a sandwich assay) preferentially or specifically binding the short VEGF-A isoforms, VEGF₁₂₁ and VEGF₁₁₀, respectively as compared to the longer naturally occurring VEGF-A isoforms VEGF₁₆₅ and VEGF₁₈₉, respectively. Preferably the short isoforms are detected with an at least 3-fold higher sensitivity as compared to the longer isoforms. An at least 3-fold higher sensitivity is acknowledged if a standard curve is established using a short isoform (purity at least 90% by SDS-PAGE and concentration determined by OD 280 nm) and for a long isoform at a predetermined concentration (purity at least 90% by SDS-PAGE and concentration determined by OD 280 nm) using the same reagents and the same standard curve the value read of the standard curves is only one third or less of the expected concentration. Also preferred the sensitivity for the short isoforms is at least 4-fold, 5-fold, 6-fold, 7-fold, 8-fold or 9-fold higher as compared to the long isoforms, especially as compared to VEGF₁₆₅.

In one embodiment both short isoforms VEGF₁₂₁ and VEGF₁₁₀ are specifically detected. Such specific detection is e.g. possible if antibodies, especially monoclonal antibodies are used and employed that bind to the sequence generated by joining exons 4 and 8 in VEGF₁₂₁ or the free C-terminal end of VEGF₁₁₀, respectively. Such VEGF₁₁₀ anti C-terminus antibody does not bind to any VEGF-A isoform comprising amino acid 110 as part of a longer polypeptide chain or to shorter VEGF-A fragments ending e.g. at amino acid 109. The monoclonal antibody that binds to the sequence generated by joining exons 4 and 8, respectively, in VEGF₁₂₁ will not bind to the amino acid sequences comprised in the longer VEGF isoforms 165 and 189, respectively, since therein other amino acid sequences are present due to the joining of exon 4 and exon 7, and of exon 4 and exon 5, respectively (see: Ferrara, N., Mol. Biol. of the Cell 21 (2010) 687-690). Specific binding in the above sense is acknowledged, if the antibody used exhibits less than 10% cross-reactivity with a shorter fragment and less than 10% cross-reactivity with those VEGF-A isoforms not having a free C-terminal amino 110 in case of the anti-VEGF₁₁₀ antibody, or those isoforms not comprising the sequence generated by joining exons 4 and 8 in case of the anti-VEGF₁₂₁ antibody, respectively. Also preferred the cross-reactivity will be less than 5%, 4%, 3%, 2% and 1%, respectively, for both shorter fragments and not having a free C-terminal amino acid 110 or VEGF isoforms not having the sequence generated by joining exons 4 and 8, respectively.

Appropriate specific antibodies only binding the short VEGF isoforms VEGF₁₂₁ or VEGF₁₁₀, respectively, can be obtained according to standard procedures. Usually a peptide representing or comprising the C-terminal most at least 4, 5, 6, 7, 8, 9, 10 or more amino acids of VEGF₁₁₀ or a peptide representing or comprising at least 5, 6, 7, 8, 9, 10 or more amino acids comprising amino acids C-terminal and N-terminal to amino acid 115 of VEGF₁₂₁, respectively, will be synthesized, optionally coupled to a carrier and used for immunization. Specific polyclonal antibodies can be obtained by appropriate immunosorption steps. Monoclonal antibodies can easily be screened for reactivity with VEGF₁₂₁ or VEGF₁₁₀, respectively, and appropriate low cross-reactivity. Low cross-reactivity in terms of the VEGF110-specific antibody can be assessed for both shorter fragments of VEGF₁₁₀ (e.g. lacking the C-terminal amino acid of VEGF₁₁₀) and VEGF-A isoforms not having a free C-terminal amino acid of VEGF₁₁₀. Low cross-reactivity in terms of the VEGF₁₂₁-specific antibody can be assessed using VEGF-isoforms containing the amino acid sequences formed upon joining of exon 4 and exon 7, and of exon 4 and exon 5, respectively.

VEGF₁₁₁ protein or nucleic acids can be detected using any method known in the art. For example, tissue or cell samples from mammals can be conveniently assayed for, e.g., proteins using Westerns, ELISAs, mRNAs or DNAs from a genetic biomarker of interest using Northern, dot-blot, or polymerase chain reaction (PCR) analysis, array hybridization, RNase protection assay, or using DNA SNP chip microarrays, which are commercially available, including DNA microarray snapshots. For example, real-time PCR (RT-PCR) assays such as quantitative PCR assays are well known in the art. In an illustrative embodiment of the invention, a method for detecting mRNA from a genetic biomarker of interest in a biological sample comprises producing cDNA from the sample by reverse transcription using at least one primer; amplifying the cDNA so produced; and detecting the presence of the amplified cDNA. In addition, such methods can include one or more steps that allow one to determine the levels of mRNA in a biological sample (e.g., by simultaneously examining the levels a comparative control mRNA sequence of a “housekeeping” gene such as an actin family member). Optionally, the sequence of the amplified cDNA can be determined.

Many references are available to provide guidance in applying the above techniques (Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987).

As to detection of VEGF₁₁₁ protein, various assays are available For example, the sample may be contacted with an antibody or an antibody combination (e.g. in a sandwich assay) preferentially or specifically binding to VEGF₁₁₁ as compared to the longer naturally occurring VEGF-A isoforms VEGF₁₆₅ and VEGF₁₈₉, respectively. Preferably the short isoform VEGF₁₁₁ is detected with an at least 3-fold higher sensitivity as compared to the longer isoforms. An at least 3-fold higher sensitivity is acknowledged if a standard curve is established using a short isoform (purity at least 90% by SDS-PAGE and concentration determined by OD 280 nm) and for a long isoform at a predetermined concentration (purity at least 90% by SDS-PAGE and concentration determined by OD 280 nm) using the same reagents and the same standard curve the value read of the standard curves is only one third or less of the expected concentration. Also preferred the sensitivity for the short isoforms is at least 4-fold, 5-fold, 6-fold, 7-fold, 8-fold or 9-fold higher as compared to the long isoforms.

In one embodiment isoform VEGF₁₁₁ is specifically detected. Such specific detection is e.g. possible if antibodies, especially monoclonal antibodies are used and employed that bind to the exon junction unique for VEGF₁₁₁. Such antibody does not bind to other VEGF-A isoform or cleavage products thereof not comprising this specific exon junction. Specific binding in the above sense is acknowledged, if the antibody used exhibits less than 10% cross-reactivity with other VEGF-A isoforms, like VEGF₁₂₁ or VEGF₁₆₅, respectively, not having this unique exon junction. Also preferred the cross-reactivity to e.g. VEGF₁₂₁ will be less than 5%, 4%, 3%, 2% and 1%, respectively.

Specificity for VEGF₁₁₁ in one embodiment is assessed by comparing VEGF111 (purity at least 90% by SDS-PAGE and concentration determined by OD 280 nm) and VEGF121 (purity at least 90% by SDS-PAGE and concentration determined by OD 280 nm) using the same reagents. If in this comparison the signal obtained for VEGF₁₂₁ material is only one tens or less of the signal as obtained with the VEGF₁₁₁ material, then cross-reactivity towards VEGF₁₂₁ is less than 10%. As the skilled artisan will appreciate the VEGF₁₂₁ signal is preferably read of at a concentration which yields about 50% of the maximal signal for VEGF₁₁₁.

Appropriate specific antibodies only binding the short VEGF isoform VEGF₁₁₁ can be obtained according to standard procedures. Usually a peptide representing or comprising amino acids C-terminal and N-terminal to amino acid 105 of VEGF₁₁₁ will be synthesized, optionally coupled to a carrier and used for immunization. Preferably such peptide will be at least six amino acids long and comprise at least the amino acids 105 and 106 of VEGF₁₁₁. Also preferred it will comprise at least the amino acids 104, 105, 106 and 107 of VEGF₁₁₁. As the skilled artisan will appreciate longer peptides comprising e.g. 3 or more amino acids N- and C-terminal to the exon junction between amino acids 105 and 106 of VEGF₁₁₁ can also be used to obtain antibodies specifically binding VEGF₁₁₁.

Unmodified VEGF protein can be detected using any appropriate method known in the art. Preferably an antibody will be used having at least the preferential binding properties to unmodified VEGF as compared to modified VEGF as MAB 3C5, which is commercially available from RELIATech GmbH, Wolfenbüttel, Germany. For example, tissue or cell samples from mammals can be conveniently assayed for the unmodified VEGF protein using Westerns, ELISAs, etc. Many references are available to provide guidance in applying the above techniques (Kohler et al., Hybridoma Techniques (Cold Spring Harbor Laboratory, New York, 1980); Tijssen, Practice and Theory of Enzyme Immunoassays (Elsevier, Amsterdam, 1985); Campbell, Monoclonal Antibody Technology (Elsevier, Amsterdam, 1984); Hurrell, Monoclonal Hybridoma Antibodies: Techniques and Applications (CRC Press, Boca Raton, Fla., 1982); and Zola, Monoclonal Antibodies: A Manual of Techniques, pp. 147-1 58 (CRC Press, Inc., 1987)).

If reference is made to the detection or level of unmodified VEGF this means that unmodified VEGF-molecules (isoforms or cleavage products) as e.g. bound by MAB 3C5 are measured.

As to detection of unmodified VEGF protein, various assays are available. For example, the sample may be contacted with an antibody or an antibody combination (e.g. in a sandwich assay) preferentially or specifically binding to unmodified VEGF as compared to modified VEGF, e.g. as naturally occurring in a patient's sample. Preferably unmodified VEGF is detected using an antibody specifically binding to unmodified VEGF, i.e., with an antibody having at least 3-fold higher sensitivity for unmodified VEGF165 as compared to modified VEGF165. Such at least 3-fold higher sensitivity for unmodified VEGF is assessed by comparing VEGF165 recombinantly produced in E. coli (purity at least 90% by SDS-PAGE and concentration determined by OD 280 nm) and VEGF165 recombinantly produced in HEK cells (purity at least 90% by SDS-PAGE and concentration determined by OD 280 nm) using the same reagents. If in this comparison the signal obtained for the HEK-produced material is only one third or less of the signal as obtained with the E. coli-derived material, then unmodified VEGF is detected with an at least 3-fold higher sensitivity. As the skilled artisan will appreciate the signal is preferably read of at about 50% of the maximal signal. Preferably in this assessment the assay of example 5 is used. Also preferred the antibody specifically binding to unmodified VEGF (VEGF165 ex E. coli) is an antibody that detects unmodified VEGF with and at least 4-fold, 5-fold, 6-fold, 7-fold, 8-fold, 9-fold or 10-fold higher sensitivity as compared to the modified VEGF material (VEGF165 ex HEK cells).

In one embodiment unmodified VEGF is specifically detected using an antibody having at least the same binding preference for unmodified VEGF as compared to modified VEGF as the commercially available MAB 3C5. In one embodiment the relative sensitivity for or preferential binding of an antibody to unmodified VEGF is assessed in a sandwich immuno assay, wherein the antibody to unmodified VEGF is used as a capture antibody and a detection antibody is used that binds to an epitope present on all major VEGF-isoforms or cleavage products. In one embodiment the detection antibody will bind to an epitope outside the epitope for MAB 3C5, i.e., it will not bind to an epitope comprised in a synthetic peptide spanning amino acids 33 to 43 of VEGF. Preferably the detection antibody will bind to an epitope comprised in the amino acids ranging from 1 to 32, form 44 to 105, to the last six amino acids of mature VEGF165, or to a conformational epitope not overlapping with the epitope bound by MAB 3C5. In one embodiment the antibody specifically binding unmodified VEGF165 as compared to modified VEGF has the property to bind to an epitope comprised in a synthetic peptide spanning amino acids 33 to 43 of VEGF.

Appropriate specific antibodies specifically binding unmodified VEGF can be obtained according to standard procedures. Usually an isoform of VEGF produced recombinantly in E. coli or obtained synthetically e.g. by solid phase polypeptide synthesis, or a peptide representing or comprising an epitope of VEGF produced recombinantly in E. coli or obtained synthetically e.g. by solid phase polypeptide synthesis will be used as an immunogen. Monoclonal antibodies can easily be produced according to standard protocols and screened for reactivity with unmodified VEGF and appropriate low cross-reactivity with modified VEGF. One convenient and preferred screening method is based on the use of VEGF165 recombinantly produced in E. coli (purity at least 90% by SDS-PAGE and concentration determined by OD 280 nm) and of VEGF165 recombinantly produced in HEKcells (purity at least 90% by SDS-PAGE and concentration determined by OD 280 nm), respectively.

The expression level of one or more of VEGFA, VEGFR2 and PLGF may be assessed in a patient sample that is a biological sample. The patient sample may be a blood sample, blood serum sample or a blood plasma sample. In one embodiment, the sample is EDTA-plasma. In one embodiment, the sample is citrate-plasma. Methods of obtaining blood samples, blood serum samples and blood plasma samples are well known in the art. The patient sample may be obtained from the patient prior to or after neoadjuvant therapy or prior to or after adjuvant therapy.

In the context of the present invention, bevacizumab is to be administered in addition to or as a co-therapy or co-treatment with one or more chemotherapeutic agents administered as part of standard chemotherapy regimen as known in the art. Examples of agents included in such standard chemotherapy regimens include 5-fluorouracil, leucovorin, irinotecan, gemcitabine, erlotinib, capecitabine, taxanes, such as docetaxel and paclitaxel, interferon alpha, vinorelbine, and platinum-based chemotherapeutic agents, such as, carboplatin, cisplatin and oxaliplatin. As demonstrated in the appended illustrative example, the addition of bevacizumab to gemcitabine-erlotinib therapy effected an increase in the overall survival and/or progression free survival in the patients and/or patient population defined and selected according to the expression level of one or more of VEGFA, VEGFR2 and PLGF. Thus, bevacizumab may be combined with a chemotherapy regimen, such as gemcitabine-erlotinib therapy as demonstrated in the appended illustrative example.

Common modes of administration include parenteral administration as a bolus dose or as an infusion over a set period of time, e.g., administration of the total daily dose over 10 min., 20 min, 30 min, 40 min, 50 min., 60 min., 75 min., 90 min, 105 min, 120 min, 3 hr., 4 hr., 5 hr. or 6 hr. For example, 2.5 mg/kg of body weight to 15 mg/kg of body weight bevacizumab (Avastin®) can be administered every week, every 2 weeks or every 3 weeks, depending on the type of cancer being treated. Examples of dosages include 2.5 mg/kg of body weight, 5 mg/kg of body weight, 7.5 mg/kg of body weight, 10 mg/kg of body weight and 15 mg/kg of body weight given every week, every 2 weeks or every 3 weeks. Further examples of dosages are 5 mg/kg of body weight every 2 weeks, 10 mg/kg every 2 weeks of body weight, 7.5 mg/kg of body weight every 3 weeks and 15 mg/kg of body weight every 3 weeks. For the treatment of pancreatic cancer, in particular metastatic pancreatic cancer, dosages include 5 mg/kg of body weight every 2 weeks, 10 mg/kg every 2 weeks, 7.5 mg/kg of body weight every 3 weeks and 15 mg/kg of body weight every 3 weeks. The skilled person will recognize that further modes of administration of bevacizumab are encompassed by the invention as determined by the specific patient and chemotherapy regimen, and that the specific mode of administration and therapeutic dosage are best determined by the treating physician according to methods known in the art.

The patients selected according to the methods of the present invention are treated with bevacizumab in combination with a chemotherapy regimen, and may be further treated with one or more additional anti-cancer therapies. In certain aspects, the one or more additional anti-cancer therapy is radiation.

The present invention also relates to a diagnostic composition or kit comprising oligonucleotides or polypeptides suitable for the determination of expression levels of one or more of VEGFA, VEGFR2 and PLGF. As detailed herein, oligonucleotides such as DNA, RNA or mixtures of DNA and RNA probes may be of use in detecting mRNA levels of the marker/indicator proteins, while polypeptides may be of use in directly detecting protein levels of the marker/indicator proteins via specific protein-protein interaction. In preferred aspects of the invention, the polypeptides encompassed as probes for the expression levels of one or more of VEGFA, VEGFR2 and PLGF, and included in the kits or diagnostic compositions described herein, are antibodies specific for these proteins, or specific for homologues and/or truncations thereof.

Accordingly, in a further embodiment of the present invention provides a kit useful for carrying out the methods herein described, comprising oligonucleotides or polypeptides capable of determining the expression level of one or more of VEGFA, VEGFR2 and PLGF. The oligonucleotides may comprise primers and/or probes specific for the mRNA encoding one or more of the markers/indicators described herein, and the polypeptides comprise proteins capable of specific interaction with the marker/indicator proteins, e.g., marker/indicator specific antibodies or antibody fragments.

Accordingly, the present invention relates to bevacizumab for use in an improved chemotherapy regimen for a patient suffering from pancreatic cancer wherein the expression level of one or more of VEGFA, VEGFR2 and PLGF in a patient sample in determined whereby a patient having an increased level of one or more of VEGFA VEGFR2 and PLGF relative to control levels determined in patients diagnosed with pancreatic cancer is administered bevacizumab in addition to the chemotherapy regimen.

The following similar uses can be applied mutatis mutandis.

The present invention relates to the use of bevacizumab for improving the treatment effect of a chemotherapy regimen of a patient suffering from pancreatic cancer comprising the following steps:
(a) determining the expression level of one or more of VEGFA, VEGFR2 and PLGF in a patient sample; and
(b) administering bevacizumab in combination with the chemotherapy regimen to the patient having an increased level of one or more of VEGFA VEGFR2 and PLGF relative to control levels determined in patients diagnosed with pancreatic cancer.

The pancreatic cancer may be metastatic pancreatic cancer. The chemotherapy regimen may be gemcitabine-erlotinib therapy.

Accordingly, the present invention relates to the use of bevacizumab for improving the treatment effect of a chemotherapy regimen of a patient suffering from pancreatic cancer comprising the following steps:
(a) obtaining a sample from said patient;
(b) determining the expression level of one or more of VEGFA, VEGFR2 and PLGF; and
(c) administering bevacizumab in combination with the chemotherapy regimen to the patient having an increased level of one or more of VEGFA, VEGFR2 and PLGF relative to control levels determined in patients diagnosed pancreatic cancer.

The pancreatic cancer may be metastatic pancreatic cancer. The chemotherapy regimen may be gemcitabine-erlotinib therapy.

The present invention relates to the use of bevacizumab for improving the overall survival of a patient suffering from pancreatic cancer comprising the following steps:
(a) determining the expression level of one or more of VEGFA, VEGFR2 and PLGF in a patient sample; and
(b) administering bevacizumab in combination with a chemotherapy regimen to the patient having an increased level of one or more of VEGFA VEGFR2 and PLGF relative to control levels determined in patients diagnosed with pancreatic cancer.

The pancreatic cancer may be metastatic pancreatic cancer. The chemotherapy regimen may be gemcitabine-erlotinib therapy.

Accordingly, the present invention relates to the use of bevacizumab for improving the overall survival of a patient suffering from pancreatic cancer comprising the following steps:
(a) obtaining a sample from said patient;
(b) determining the expression level of one or more of VEGFA, VEGFR2 and PLGF; and
(c) administering bevacizumab in combination with a chemotherapy regimen to the patient having an increased level of one or more of VEGFA, VEGFR2 and PLGF relative to control levels determined in patients diagnosed pancreatic cancer.

The pancreatic cancer may be metastatic pancreatic cancer. The chemotherapy regimen may be gemcitabine-erlotinib therapy.

The present invention relates to the use of bevacizumab for improving the progression free survival of a patient suffering from pancreatic cancer comprising the following steps:
(a) determining the expression level of one or more of VEGFA, VEGFR2 and PLGF in a patient sample; and
(b) administering bevacizumab in combination with the chemotherapy regimen to the patient having an increased level of one or more of VEGFA VEGFR2 and PLGF relative to control levels determined in patients diagnosed with pancreatic cancer.

The pancreatic cancer may be metastatic pancreatic cancer. The chemotherapy regimen may be gemcitabine-erlotinib therapy.

Accordingly, the present invention relates to the use of bevacizumab for improving the progression free survival of a patient suffering from pancreatic cancer comprising the following steps:
(a) obtaining a sample from said patient;
(b) determining the expression level of one or more of VEGFA, VEGFR2 and PLGF; and
(c) administering bevacizumab in combination with the chemotherapy regimen to the patient having an increased level of one or more of VEGFA, VEGFR2 and PLGF relative to control levels determined in patients diagnosed pancreatic cancer. The pancreatic cancer may be metastatic pancreatic cancer. The chemotherapy regimen may be gemcitabine-erlotinib therapy.

The present invention relates to the use of bevacizumab for improving the overall survival of a patient suffering from pancreatic cancer comprising the following steps:
(a) determining the expression level of VEGFA or VEGFR2 in a patient sample; and
(b) administering bevacizumab in combination with a chemotherapy regimen to the patient having an increased level of VEGFA or VEGFR2 relative to control levels determined in patients diagnosed with pancreatic cancer.

The pancreatic cancer may be metastatic pancreatic cancer. The chemotherapy regimen may be gemcitabine-erlotinib therapy.

Accordingly, the present invention relates to the use of bevacizumab for improving the overall survival of a patient suffering from pancreatic cancer comprising the following steps:
(a) obtaining a sample from said patient;
(b) determining the expression level of VEGFA or VEGFR2; and
(c) administering bevacizumab in combination with a chemotherapy regimen to the patient having an increased level of VEGFA or VEGFR2 relative to control levels determined in patients diagnosed pancreatic cancer.

The pancreatic cancer may be metastatic pancreatic cancer. The chemotherapy regimen may be gemcitabine-erlotinib therapy.

The present invention relates to the use of bevacizumab for improving the progression free survival of a patient suffering from pancreatic cancer comprising the following steps:
(a) determining the expression level of VEGFA or PLGF in a patient sample; and
(b) administering bevacizumab in combination with the chemotherapy regimen to the patient having an increased level of VEGFA or PLGF relative to control levels determined in patients diagnosed with pancreatic cancer.

The pancreatic cancer may be metastatic pancreatic cancer. The chemotherapy regimen may be gemcitabine-erlotinib therapy.

Accordingly, the present invention relates to the use of bevacizumab for improving the progression free survival of a patient suffering from pancreatic cancer comprising the following steps:
(a) obtaining a sample from said patient;
(b) determining the expression level of VEGFA or PLGF; and
(c) administering bevacizumab in combination with the chemotherapy regimen to the patient having an increased level of VEGFA or PLGF relative to control levels determined in patients diagnosed pancreatic cancer.

The pancreatic cancer may be metastatic pancreatic cancer. The chemotherapy regimen may be gemcitabine-erlotinib therapy.

The present invention provides the use of bevacizumab for improving overall survival of a patient suffering from metastatic pancreatic cancer comprising the following steps:
(a) determining the expression level of VEGFA and VEGFR2 in a patient sample; and
(b) administering bevacizumab in combination with a chemotherapy regimen to the patient having an increased combined expression level of VEGFA and VEGFR2 relative to a control combined expression level determined in patients diagnosed with metastatic pancreatic cancer.

The present invention relates to the use of bevacizumab for improving overall survival of a patient suffering from metastatic pancreatic cancer comprising the following steps:
(a) obtaining a sample from said patient;
(b) determining the expression level of VEGFA and VEGFR2; and
(c) administering bevacizumab in combination with a chemotherapy regimen to the patient having an increased combined expression level of VEGFA and VEGFR2 relative to a control combined expression level determined in patients diagnosed with metastatic pancreatic cancer.

The invention relates to the use of bevacizumab for improving overall survival of a patient suffering from metastatic pancreatic cancer comprising the following steps:
(a) determining the expression level of VEGFA and VEGFR2 in a patient sample; and
(b) administering bevacizumab in combination with a chemotherapy regimen to the patient having an increased combined expression level of VEGFA and VEGFR2 relative to a control combined expression level determined in patients diagnosed with metastatic pancreatic cancer,
   wherein the chemotherapy regimen is gemcitabine-erlotinib therapy.

Accordingly, the present invention relates to the use of bevacizumab for overall survival of a patient suffering from metastatic pancreatic cancer comprising the following steps:
(a) obtaining a sample from said patient;
(b) determining the expression level of VEGFA and VEGFR2; and
(c) administering bevacizumab in combination with a chemotherapy regimen to the patient having an increased combined expression level of VEGFA and VEGFR2 relative to a control combined expression level determined in patients diagnosed with metastatic pancreatic cancer,
   wherein the chemotherapy regimen is gemcitabine-erlotinib therapy.

The present invention provides the use of bevacizumab for improving progression free survival of a patient suffering from metastatic pancreatic cancer comprising the following steps:
(a) determining the expression level of VEGFA and VEGFR2 in a patient sample; and
(b) administering bevacizumab in combination with a chemotherapy regimen to the patient having an increased combined expression level of VEGFA and VEGFR2 relative to a control combined expression level determined in patients diagnosed with metastatic pancreatic cancer.

The present invention relates to the use of bevacizumab for improving progression free survival of a patient suffering from metastatic pancreatic cancer comprising the following steps:
(a) obtaining a sample from said patient;
(b) determining the expression level of VEGFA and VEGFR2; and
(c) administering bevacizumab in combination with a chemotherapy regimen to the patient having an increased combined expression level of VEGFA and VEGFR2 relative to a control combined expression level determined in patients diagnosed with metastatic pancreatic cancer.

The invention relates to the use of bevacizumab for improving progression free survival of a patient suffering from metastatic pancreatic cancer comprising the following steps:
(a) determining the expression level of VEGFA and VEGFR2 in a patient sample; and
(b) administering bevacizumab in combination with a chemotherapy regimen to the patient having an increased combined expression level of VEGFA and VEGFR2 relative to a control combined expression level determined in patients diagnosed with metastatic pancreatic cancer,
   wherein the chemotherapy regimen is gemcitabine-erlotinib therapy.

Accordingly, the present invention relates to the use of bevacizumab for progression free survival of a patient suffering from metastatic pancreatic cancer comprising the following steps:
(a) obtaining a sample from said patient;
(b) determining the expression level of VEGFA and VEGFR2; and
(c) administering bevacizumab in combination with a chemotherapy regimen to the patient having an increased combined expression level of VEGFA and VEGFR2 relative to a control combined expression level determined in patients diagnosed with metastatic pancreatic cancer,
   wherein the chemotherapy regimen is gemcitabine-erlotinib therapy.

The present invention provides the use of bevacizumab for improving overall survival of a patient suffering from metastatic pancreatic cancer comprising the following steps:
(a) determining the expression level of VEGFA and PLGF in a patient sample; and
(b) administering bevacizumab in combination with a chemotherapy regimen to the patient having an increased combined expression level of VEGFA and PLGF relative to a control combined expression level determined in patients diagnosed with metastatic pancreatic cancer.

Accordingly, the present invention relates to the use of bevacizumab for overall survival of a patient suffering from metastatic pancreatic cancer comprising the following steps:
(a) obtaining a sample from said patient;
(b) determining the expression level of VEGFA and PLGF; and
(c) administering bevacizumab in combination with a chemotherapy regimen to the patient having an increased combined expression level of VEGFA and PLGF relative to a control combined expression level determined in patients diagnosed with metastatic pancreatic cancer.

The present invention provides the use of bevacizumab for improving overall survival of a patient suffering from metastatic pancreatic cancer comprising the following steps:
(a) determining the expression level of VEGFA and PLGF in a patient sample; and
(b) administering bevacizumab in combination with a chemotherapy regimen to the patient having an increased combined expression level of VEGFA and PLGF relative to a control combined expression level determined in patients diagnosed with metastatic pancreatic cancer,
   wherein the chemotherapy regimen is gemcitabine-erlotinib therapy.

Accordingly, the present invention relates to the use of bevacizumab for overall survival of a patient suffering from metastatic pancreatic cancer comprising the following steps:
(a) obtaining a sample from said patient;
(b) determining the expression level of VEGFA and PLGF; and
(c) administering bevacizumab in combination with a chemotherapy regimen to the patient having an increased combined expression level of VEGFA and PLGF relative to a control combined expression level determined in patients diagnosed with metastatic pancreatic cancer
   wherein the chemotherapy regimen is gemcitabine-erlotinib therapy.

The present invention provides the use of bevacizumab for improving progression free survival of a patient suffering from metastatic pancreatic cancer comprising the following steps:
(a) determining the expression level of VEGFA and PLGF in a patient sample; and
(b) administering bevacizumab in combination with a chemotherapy regimen to the patient having an increased combined expression level of VEGFA and PLGF relative to a control combined expression level determined in patients diagnosed with metastatic pancreatic cancer.

Accordingly, the present invention relates to the use of bevacizumab for progression free survival of a patient suffering from metastatic pancreatic cancer comprising the following steps:
(a) obtaining a sample from said patient;
(b) determining the expression level of VEGFA and PLGF; and
(c) administering bevacizumab in combination with a chemotherapy regimen to the patient having an increased combined expression level of VEGFA and PLGF relative to a control combined expression level determined in patients diagnosed with metastatic pancreatic cancer.

The present invention provides the use of bevacizumab for improving progression free survival of a patient suffering from metastatic pancreatic cancer comprising the following steps:
(a) determining the expression level of VEGFA and PLGF in a patient sample; and
(b) administering bevacizumab in combination with a chemotherapy regimen to the patient having an increased combined expression level of VEGFA and PLGF relative to a control combined expression level determined in patients diagnosed with metastatic pancreatic cancer,
   wherein the chemotherapy regimen is gemcitabine-erlotinib therapy.

Accordingly, the present invention relates to the use of bevacizumab for progression free survival of a patient suffering from metastatic pancreatic cancer comprising the following steps:
(a) obtaining a sample from said patient;
(b) determining the expression level of VEGFA and PLGF; and
(c) administering bevacizumab in combination with a chemotherapy regimen to the patient having an increased combined expression level of VEGFA and PLGF relative to a control combined expression level determined in patients diagnosed with metastatic pancreatic cancer
   wherein the chemotherapy regimen is gemcitabine-erlotinib therapy.

The present invention provides the use of bevacizumab for improving overall survival of a patient suffering from metastatic pancreatic cancer comprising the following steps:
(a) determining the expression level of VEGFA, VEGFR2 and PLGF in a patient sample; and
(b) administering bevacizumab in combination with a chemotherapy regimen to the patient having an increased combined expression level of VEGFA, VEGFR2 and PLGF relative to a control combined expression level determined in patients diagnosed with metastatic pancreatic cancer.

The present invention relates to the use of bevacizumab for improving overall survival of a patient suffering from metastatic pancreatic cancer comprising the following steps:
(a) obtaining a sample from said patient;
(b) determining the expression level of VEGFA, VEGFR2 and PLGF; and
(c) administering bevacizumab in combination with a chemotherapy regimen to the patient having an increased combined expression level of VEGFA, VEGFR2 and PLGF relative to a control combined expression level determined in patients diagnosed with metastatic pancreatic cancer.

The invention relates to the use of bevacizumab for improving overall survival of a patient suffering from metastatic pancreatic cancer comprising the following steps:
(a) determining the expression level of VEGFA, VEGFR2 and PLGF in a patient sample; and
(b) administering bevacizumab in combination with a chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen is gemcitabine-erlotinib therapy.

Accordingly, the present invention relates to the use of bevacizumab for overall survival of a patient suffering from metastatic pancreatic cancer comprising the following steps:
(a) obtaining a sample from said patient;
(b) determining the expression level of VEGFA, VEGFR2 and PLGF; and
(c) administering bevacizumab in combination with a chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen is gemcitabine-erlotinib therapy.

The present invention provides the use of bevacizumab for improving progression free survival of a patient suffering from metastatic pancreatic cancer comprising the following steps:
(a) determining the expression level of VEGFA, VEGFR2 and PLGF in a patient sample; and
(b) administering bevacizumab in combination with a chemotherapy regimen to the patient having an increased combined expression level of VEGFA, VEGFR2 and PLGF relative to a control combined expression level determined in patients diagnosed with metastatic pancreatic cancer.

The present invention relates to the use of bevacizumab for improving progression free survival of a patient suffering from metastatic pancreatic cancer comprising the following steps:
(a) obtaining a sample from said patient;
(b) determining the expression level of VEGFA, VEGFR2 and PLGF; and
(c) administering bevacizumab in combination with a chemotherapy regimen to the patient having an increased combined expression level of VEGFA, VEGFR2 and PLGF relative to a control combined expression level determined in patients diagnosed with metastatic pancreatic cancer.

The invention relates to the use of bevacizumab for improving progression free survival of a patient suffering from metastatic pancreatic cancer comprising the following steps:
(a) determining the expression level of VEGFA, VEGFR2 and PLGF in a patient sample; and
(b) administering bevacizumab in combination with a chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen is gemcitabine-erlotinib therapy.

Accordingly, the present invention relates to the use of bevacizumab for progression free survival of a patient suffering from metastatic pancreatic cancer comprising the following steps:
(a) obtaining a sample from said patient;
(b) determining the expression level of VEGFA, VEGFR2 and PLGF; and
(c) administering bevacizumab in combination with a chemotherapy regimen to the patient having an increased combined expression level of VEGFA, VEGFR2 and PLGF relative to a control combined expression level determined in patients diagnosed with metastatic pancreatic cancer,
   wherein the chemotherapy regimen is gemcitabine-erlotinib therapy.

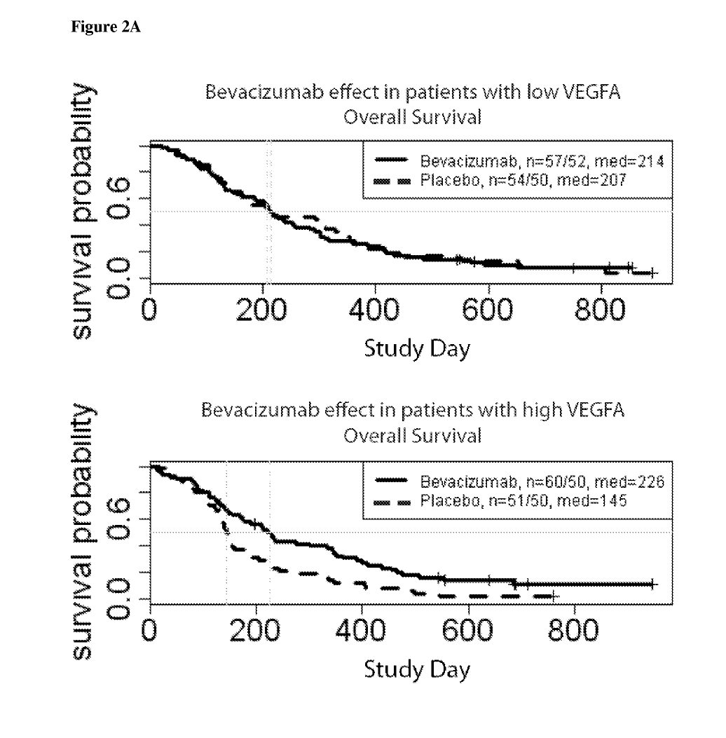

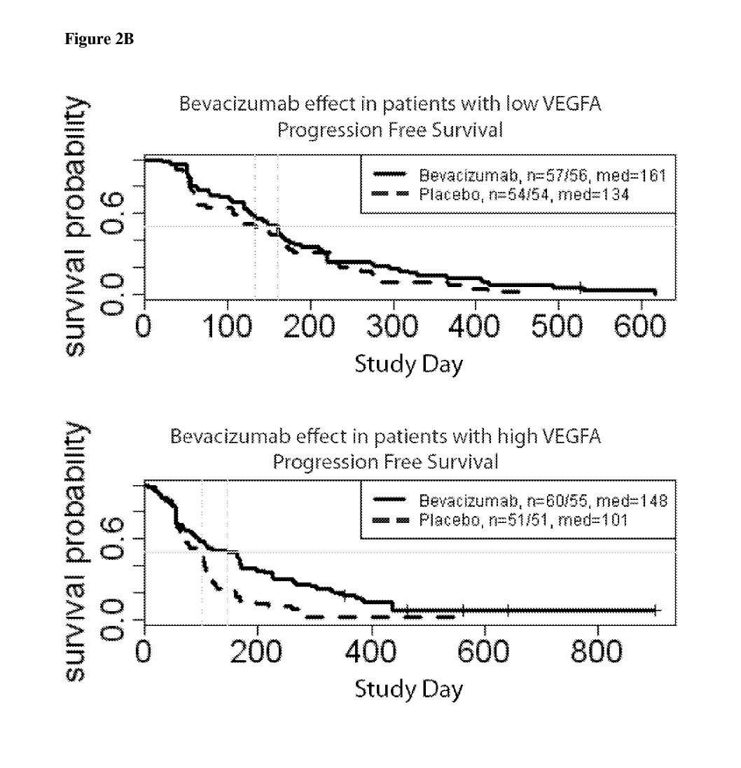

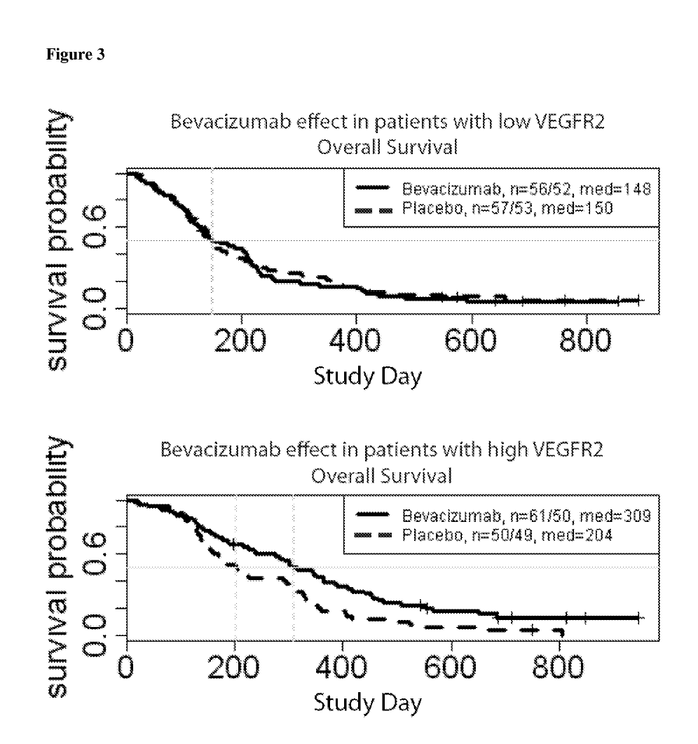

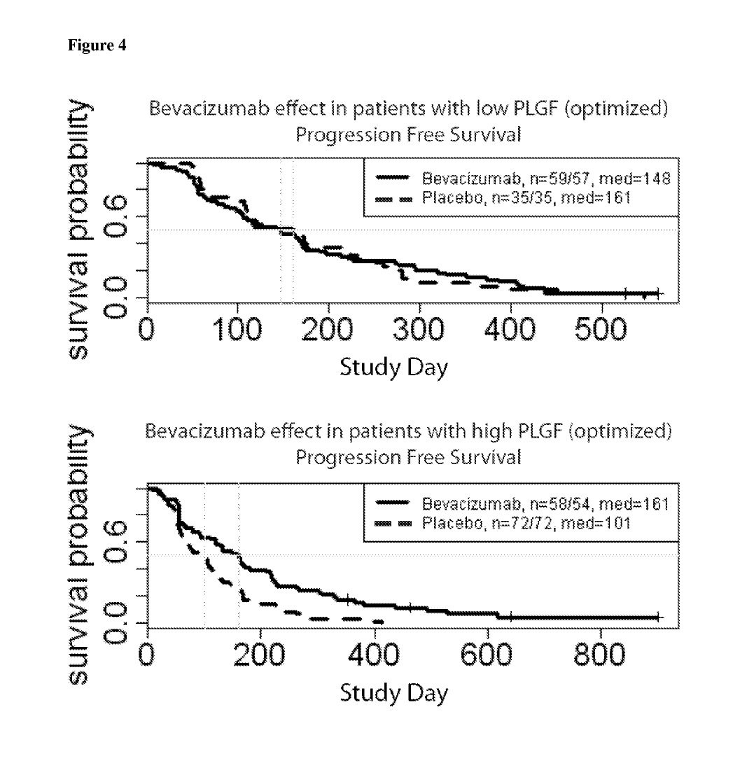

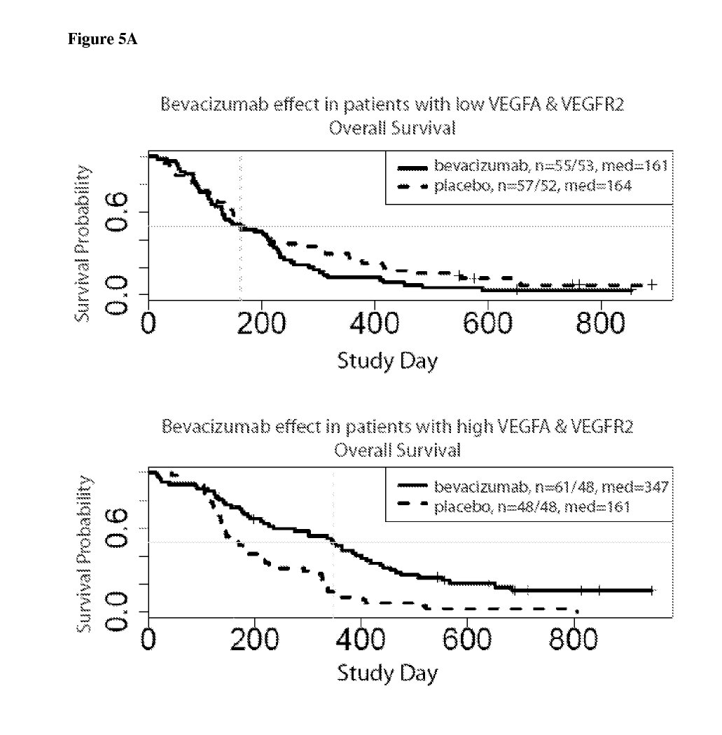

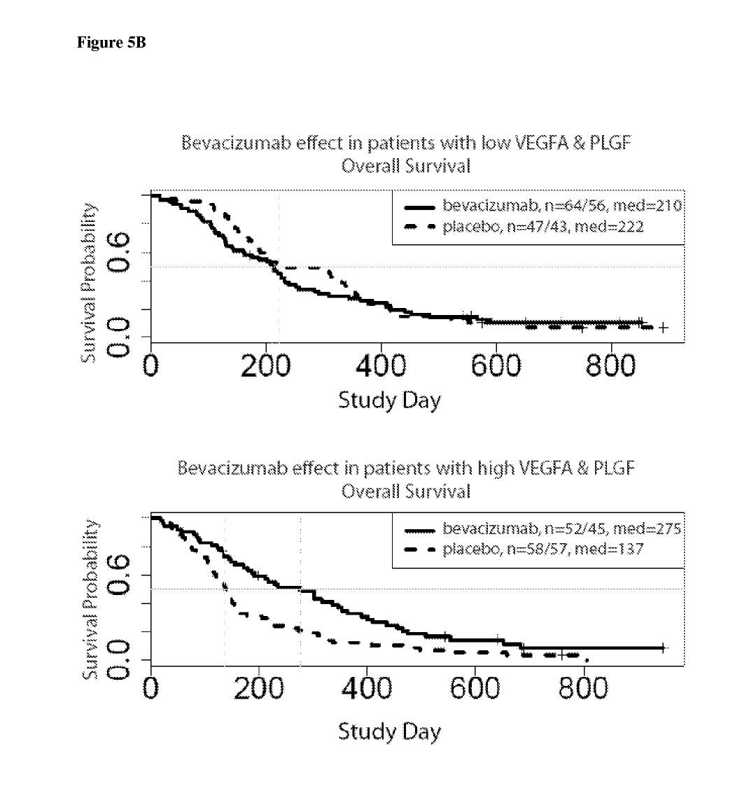

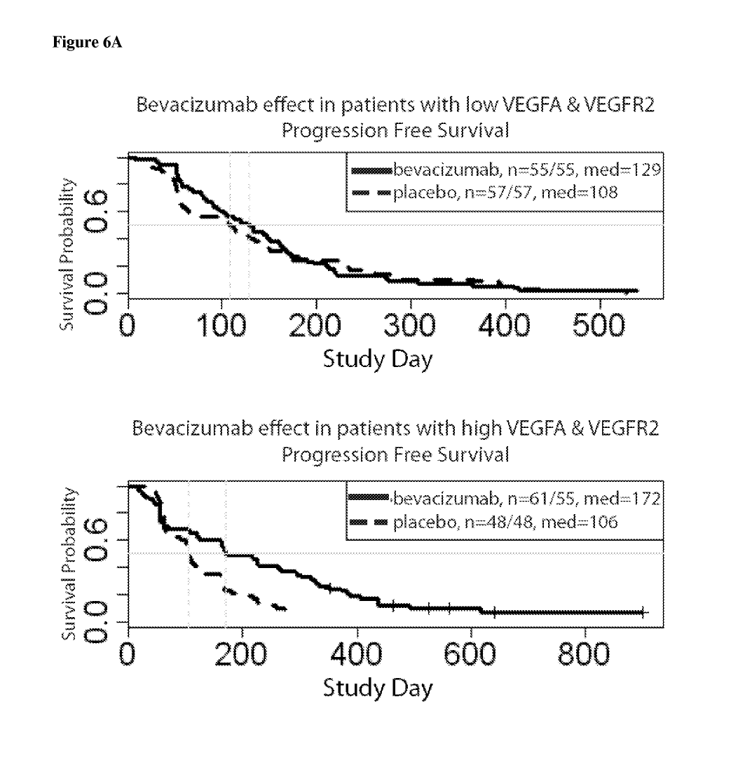

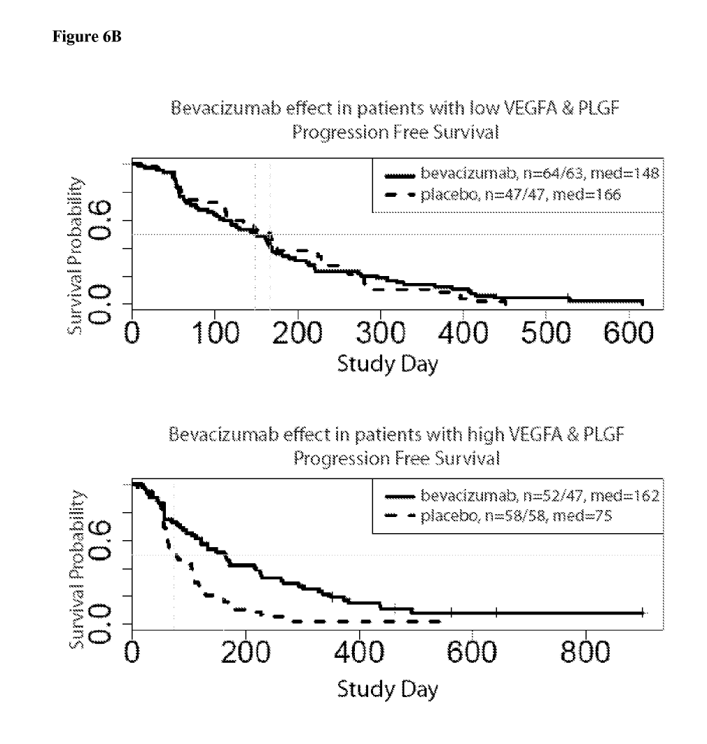

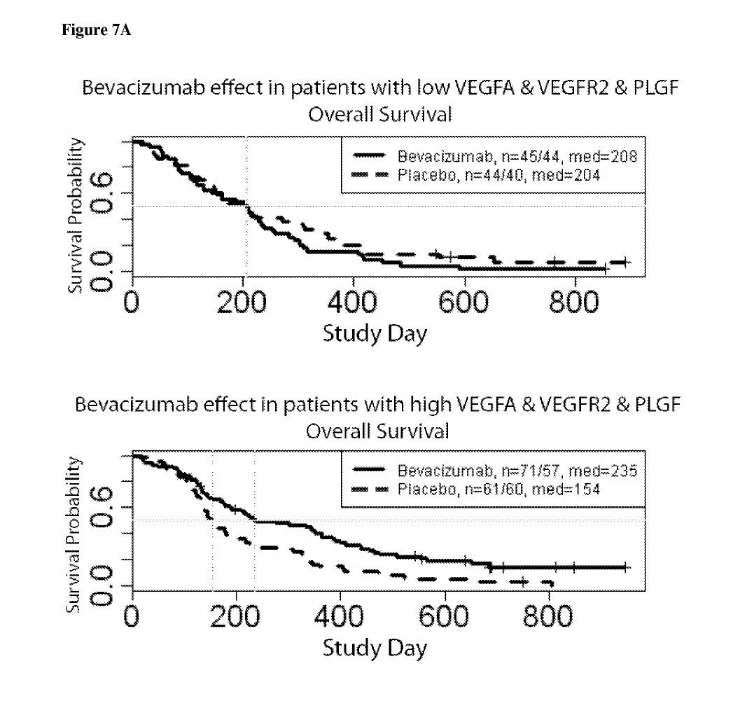

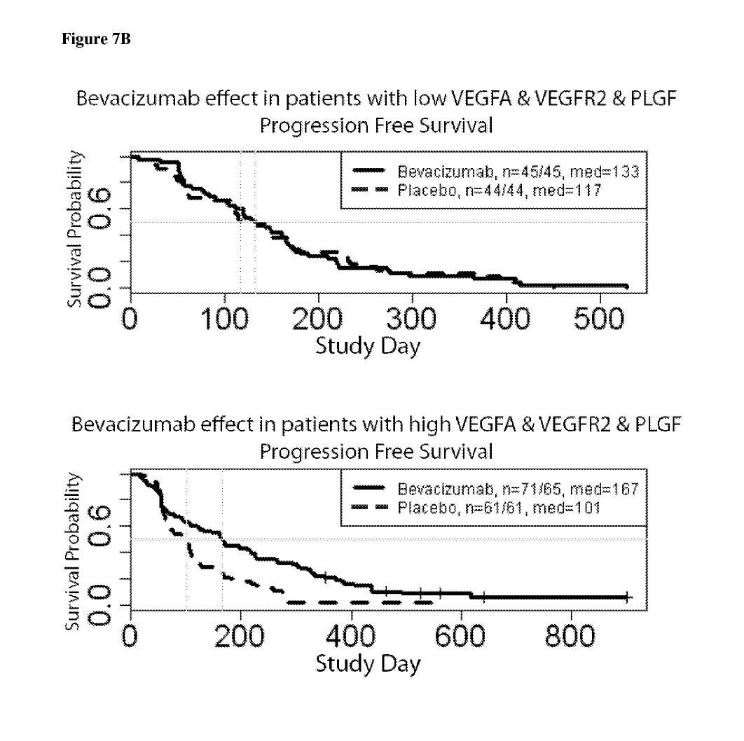

As documented in the appended illustrative example, the present invention solves the identified technical problem in that it could surprisingly be shown that the expression levels of one or more of VEGFA, VEGFR2 and PLGF in a given patient, relative to control levels determined in patients diagnosed with pancreatic cancer, in particular metastatic pancreatic cancer, correlate with treatment effect in patients administered bevacizumab in combination with a gemcitabine-erlotinib chemotherapy regimen. As is shown in the appended illustrative example, it was surprisingly found that an increased protein expression level of VEGFA or VEGFR2 correlated with improved overall survival of patients suffering from metastatic pancreatic cancer that were treated with bevacizumab and a gemcitabine-erlotinib chemotherapy regimen in comparison to patients treated with placebo and a gemcitabine-erlotinib chemotherapy regimen (FIGS. 2 and 3). It was surprisingly found that an increased protein expression level of VEGFA or PLGF correlated with improved progression free survival of patients suffering from metastatic pancreatic cancer that were treated with bevacizumab and a gemcitabine-erlotinib chemotherapy regimen in comparison to patients treated with placebo and a gemcitabine-erlotinib chemotherapy (FIGS. 2 and 4). It was further surprisingly found that an increased combined expression level of VEGFA and VEGFR2 correlated with improved overall survival and progression free survival of patients suffering from metastatic pancreatic cancer that were treated with bevacizumab and a gemcitabine-erlotinib chemotherapy regimen in comparison to patients treated with placebo and a gemcitabine-erlotinib chemotherapy regimen (FIGS. 5 and 6). It was also surprisingly found that an increased combined expression level of VEGFA and PLGF correlated with improved overall survival and progression free survival in patients suffering from metastatic pancreatic cancer that were treated with bevacizumab and a gemcitabine-erlotinib chemotherapy regimen in comparison to patients treated with placebo and a gemcitabine-erlotinib chemotherapy regimen (FIGS. 5 and 6). It was further surprisingly found that an increased combined expression level of VEGFA, VEGFR2 and PLGF correlated with improved overall survival and progression free survival in patients suffering from metastatic pancreatic cancer that were treated with bevacizumab and a gemcitabine-erlotinib chemotherapy regimen in comparison to patients treated with placebo and a gemcitabine-erlotinib chemotherapy regimen (FIG. 7). These results are particularly surprising in that these individual markers and the above described combinations of these markers showed no correlation with overall survival when analysed in patient blood plasma samples from a study comparing docetaxel therapy plus bevacizumab or placebo in patients suffering from locally advanced, recurrent or metastatic HER-2 negative breast cancer.

The invention, therefore, relates to an in vitro method of predicting the response to or sensitivity to the addition of bevacizumab to a chemotherapy regimen of a patient suffering from, suspect to suffer from or prone to suffer from pancreatic cancer, in particular metastatic pancreatic cancer, comprising determining the expression level, including combined expression levels, of one or more of VEGFA, VEGFR2 and PLGF in a patient sample. Accordingly, in the context of the methods described herein, the invention provides the use of specific probes, including for example binding molecules like antibodies and aptamers, for the preparation of a diagnostic composition for predicting the response to or sensitivity to the addition of bevacizumab to a chemotherapy regimen of a patient suffering from, suspect to suffer from or prone to suffer from pancreatic cancer, in particular metastatic pancreatic cancer, comprising determining the expression level, including combined expression levels, of one or more of VEGFA, VEGFR2 and PLGF in a patient sample.

The invention, therefore, provides an in vitro method of predicting the response to or sensitivity to the addition of bevacizumab to a chemotherapy regimen of a patient suffering from, suspected to suffer from, or prone to suffer from metastatic pancreatic cancer comprising determining the combined expression level of VEGFA and VEGFR2 in a patient sample. Accordingly, in the context of the methods described herein, the invention provides the use of specific probes, including for example binding molecules like antibodies and aptamers, for the preparation of a diagnostic composition for predicting the response to or sensitivity to the addition of bevacizumab to a chemotherapy regimen of a patient suffering from, suspect to suffer from or prone to suffer from metastatic pancreatic cancer comprising determining the combined expression level of VEGFA and VEGFR2 in a patient sample.

The invention provides an in vitro method of predicting the response to or sensitivity to the addition of bevacizumab to a chemotherapy regimen of a patient suffering from, suspected to suffer from, or prone to suffer from metastatic pancreatic cancer comprising determining the combined expression level of VEGFA and PLGF in a patient sample. Accordingly, in the context of the methods described herein, the invention provides the use of specific probes, including for example binding molecules like antibodies and aptamers, for the preparation of a diagnostic composition for predicting the response to or sensitivity to the addition of bevacizumab to a chemotherapy regimen of a patient suffering from, suspect to suffer from or prone to suffer from metastatic pancreatic cancer comprising determining the combined expression level of VEGFA and PLGF in a patient sample.

The invention provides an in vitro method of predicting the response to or sensitivity to the addition of bevacizumab to a chemotherapy regimen of a patient suffering from, suspected to suffer from, or prone to suffer from metastatic pancreatic cancer comprising determining the combined expression level of VEGFA, VEGFR2 and PLGF in a patient sample. Accordingly, in the context of the methods described herein, the invention provides the use of specific probes, including for example binding molecules like antibodies and aptamers, for the preparation of a diagnostic composition for predicting the response to or sensitivity to the addition of bevacizumab to a chemotherapy regimen of a patient suffering from, suspect to suffer from or prone to suffer from metastatic pancreatic cancer comprising determining the combined expression level of VEGFA, VEGFR2 and PLGF in a patient sample.

The phrase “responsive to” in the context of the present invention indicates that a subject/patient suffering, suspected to suffer or prone to suffer from pancreatic cancer, in particular metastatic pancreatic cancer, shows a response to a chemotherapy regimen comprising the addition of bevacizumab. A skilled person will readily be in a position to determine whether a person treated with bevacizumab according to the methods of the invention shows a response. For example, a response may be reflected by decreased suffering from the metastatic pancreatic cancer, such as a diminished and/or halted tumor growth, reduction of the size of a tumor, and/or amelioration of one or more symptoms of the cancer. Preferably, the response may be reflected by decreased or diminished indices of the metastatic conversion of the pancreatic cancer such as the prevention of the formation of metastases or a reduction of number or size of metastases (see, e.g., Eisenhauser et al., New response evaluation criteria in solid tumours: Revised RECIST guideline (version 1.1) Eur. J. Cancer 2009 45: 228-247).

The phrase “sensitive to” in the context of the present invention indicates that a subject/patient suffering, suspected to suffer or prone to suffer from pancreatic cancer, in particular metastatic pancreatic cancer, shows in some way a positive reaction to treatment with bevacizumab in combination with a chemotherapy regimen. The reaction of the patient may be less pronounced when compared to a patient “responsive to” as described hereinabove. For example, the patient may experience less suffering associated with the disease, though no reduction in tumor growth or metastatic indicator may be measured, and/or the reaction of the patient to the bevacizumab in combination with the chemotherapy regimen may be only of a transient nature, i.e., the growth of (a) tumor and/or (a) metastasis(es) may only be temporarily reduced or halted.

The phrase “a patient suffering from” in accordance with the invention refers to a patient showing clinical signs of pancreatic cancer, in particular metastatic pancreatic cancer. The phrases “suspected to suffer from”, “being susceptible to”, “prone to suffer from” or “being prone to”, in the context of metastatic pancreatic cancer, refers to an indication disease in a patient based on, e.g., a possible genetic predisposition, a pre- or eventual exposure to hazardous and/or carcinogenic compounds, or exposure to carcinogenic physical hazards, such as radiation.

The phrase “treatment effect of a chemotherapy regimen” in the context of the present invention encompasses the terms “overall survival” and “progression-free survival”.

The phrase “overall survival” in the context of the present invention refers to the length of time during and after treatment the patient survives. As the skilled person will appreciate, a patient's overall survival is improved or enhanced, if the patient belongs to a subgroup of patients that has a statistically significant longer mean survival time as compared to another subgroup of patients.

The phrase “progression-free survival” in the context of the present invention refers to the length of time during and after treatment during which, according to the assessment of the treating physician or investigator, the patient's disease does not become worse, i.e., does not progress. As the skilled person will appreciate, a patient's progression-free survival is improved or enhanced if the patient experiences a longer length of time during which the disease does not progress as compared to the average or mean progression free survival time of a control group of similarly situated patients.

The terms “administration” or “administering” as used herein mean the administration of an angiogenesis inhibitor, e.g., bevacizumab (Avastin®), and/or a pharmaceutical composition/treatment regimen comprising an angiogenesis inhibitor, e.g., bevacizumab (Avastin®), to a patient in need of such treatment or medical intervention by any suitable means known in the art for administration of a therapeutic antibody. Nonlimiting routes of administration include by oral, intravenous, intraperitoneal, subcutaneous, intramuscular, topical, intradermal, intranasal or intrabronchial administration (for example as effected by inhalation). Particularly preferred in context of this invention is parenteral administration, e.g., intravenous administration.

The term “antibody” is herein used in the broadest sense and includes, but is not limited to, monoclonal and polyclonal antibodies, multispecific antibodies (e.g., bispecific antibodies), chimeric antibodies, CDR grafted antibodies, humanized antibodies, camelized antibodies, single chain antibodies and antibody fragments and fragment constructs, e.g., F(ab′)₂ fragments, Fab-fragments, Fv-fragments, single chain Fv-fragments (scFvs), bispecific scFvs, diabodies, single domain antibodies (dAbs) and minibodies, which exhibit the desired biological activity, in particular, specific binding to one or more of VEGFA, VEGFR2 and PLGF, or to homologues, variants, fragments and/or isoforms thereof.

The term “aptamer” is herein used in the broadest sense and includes, but is not limited to, oligonucleotides, including RNA, DNA and RNA/DNA molecules, or peptide molecules, which exhibit the desired biological activity, in particular, specific binding to one or more of VEGFA, VEGFR2 and PLGF, or to homologues, variants, fragments and/or isoforms thereof.

As used herein “chemotherapy regimen” or “chemotherapeutic agent” include any active agent that can provide an anticancer therapeutic effect and may be a chemical agent or a biological agent, in particular, that are capable of interfering with cancer or tumor cells. Preferred active agents are those that act as anti-neoplastic (chemotoxic or chemostatic) agents which inhibit or prevent the development, maturation or proliferation of malignant cells. Nonlimiting examples of a chemotherapy regimen or chemotherapeutic agents include alkylating agents such as nitrogen mustards (e.g., mechlorethamine, cyclophosphamide, ifosfamide, melphalan and chlorambucil), nitrosoureas (e.g., carmustine (BCNU), lomustine (CCNU), and semustine (methyl-CCNU)), ethylenimines/methylmelamines (e.g., thriethylenemelamine (TEM), triethylene, thiophosphoramide (thiotepa), hexamethylmelamine (HMM, altretamine)), alkyl sulfonates (e.g., busulfan), and triazines (e.g., dacarbazine (DTIC)); antimetabolites such as folic acid analogs (e.g., methotrexate, trimetrexate), pyrimidine analogs (e.g., 5-fluorouracil, capecitabine, fluorodeoxyuridine, gemcitabine, cytosine arabinoside (AraC, cytarabine), 5-azacytidine, 2,2′-difluorodeoxycytidine), and purine analogs (e.g., 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA)); antimitotic drugs developed from natural products (e.g., paclitaxel, vinca alkaloids (e.g., vinblastine (VLB), vincristine, and vinorelbine), docetaxel, estramustine, and estramustine phosphate), epipodophylotoxins (e.g., etoposide, teniposide), antibiotics (e.g., actimomycin D, daunomycin (rubidomycin), daunorubicon, doxorubicin, epirubicin, mitoxantrone, idarubicin, bleomycins, plicamycin (mithramycin), mitomycinC, actinomycin), enzymes (e.g., L-asparaginase), and biological response modifiers (e.g., interferon-alpha, IL-2, G-CSF, GM-CSF); miscellaneous agents including platinum coordination complexes (e.g., cisplatin, carboplatin, oxaliplatin), anthracenediones (e.g., mitoxantrone), substituted urea (i.e., hydroxyurea), methylhydrazine derivatives (e.g., N-methylhydrazine (MIH), procarbazine), adrenocortical suppressants (e.g., mitotane (o,p′-DDD), aminoglutethimide); hormones and antagonists including adrenocorticosteroid antagonists (e.g., prednisone and equivalents, dexamethasone, aminoglutethimide), progestins (e.g., hydroxyprogesterone caproate, medroxyprogesterone acetate, megestrol acetate), estrogens (e.g., diethylstilbestrol, ethinyl estradiol and equivalents thereof); antiestrogens (e.g., tamoxifen), androgens (e.g., testosterone propionate, fluoxymesterone and equivalents thereof), antiandrogens (e.g., flutamide, gonadotropin-releasing hormone analogs, leuprolide), non-steroidal antiandrogens (e.g., flutamide), epidermal growth factor inhibitors (e.g., erlotinib, lapatinib, gefitinib) antibodies (e.g., trastuzumab), irinotecan and other agents such as leucovorin. For the treatment of pancreatic cancer, in particular metastatic pancreatic cancer, chemotherapeutic agents or chemotherapy regimens for administration with bevacizumab include gemcitabine and erlotinib and combinations thereof (see also the appended illustrative example herein provided).

In the context of the present invention, “homology” with reference to an amino acid sequence is understood to refer to a sequence identity of at least 80%, particularly an identity of at least 85%, at least 90% or at least 95% over the full length of the sequence as defined by the SEQ ID NOs provided herein. In the context of this invention, a skilled person would understand that homology covers further allelic variation(s) of the marker/indicator proteins in different populations and ethnic groups.

As used herein, the term “polypeptide” relates to a peptide, a protein, an oligopeptide or a polypeptide which encompasses amino acid chains of a given length, wherein the amino acid residues are linked by covalent peptide bonds. However, peptidomimetics of such proteins/polypeptides are also encompassed by the invention wherein amino acid(s) and/or peptide bond(s) have been replaced by functional analogs, e.g., an amino acid residue other than one of the 20 gene-encoded amino acids, e.g., selenocysteine. Peptides, oligopeptides and proteins may be termed polypeptides. The terms polypeptide and protein are used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature. The term polypeptide also refers to and encompasses the term “antibody” as used herein.

The terms “treating” and “treatment” as used herein refer to remediation of, improvement of, lessening of the severity of, or reduction in the time course of the disease or any parameter or symptom thereof. Preferably said patient is a human patient and the disease to be treated is pancreatic cancer, in particular metastatic pancreatic cancer.

The terms “assessing” or “assessment” of such a patient relates to methods of determining the expression levels of one or more of the marker/indicator proteins described herein, including VEGFA, VEGFR2 and PLGF, and/or for selecting such patients based on the expression levels of such marker/indicator proteins relative to control levels established in patients diagnosed with pancreatic cancer, in particular metastatic pancreatic cancer.

The term “expression level” as used herein refers may also refer to the concentration or amount of marker/indicator proteins of the present invention in a sample.

In addition to the methods described above, the invention also encompasses further immunoassay methods for assessing or determining the expression level of one or more of VEGFA, VEGFR2 and PLGF, such as by Western blotting and ELISA-based detection. As is understood in the art, the expression level of the marker/indicator proteins of the invention may also be assessed at the mRNA level by any suitable method known in the art, such as Northern blotting, real time PCR, and RT PCR Immunoassay- and mRNA-based detection methods and systems are well known in the art and can be deduced from standard textbooks, such as Lottspeich (Bioanalytik, Spektrum Akademisher Verlag, 1998) or Sambrook and Russell (Molecular Cloning: A Laboratory Manual, CSH Press, Cold Spring Harbor, N.Y., U.S.A., 2001). The described methods are of particular use for determining the expression levels of VEGFA, VEGFR2 and PLGF in a patient or group of patients relative to control levels established in a population diagnosed with pancreatic cancer, in particular metastatic pancreatic cancer.

The expression level of one or more of VEGFA, VEGFR2 and PLGF, can also be determined on the protein level by taking advantage of immunoagglutination, immunoprecipitation (e.g., immunodiffusion, immunelectrophoresis, immune fixation), western blotting techniques (e.g., (in situ) immuno cytochemistry, affinitychromatography, enzyme immunoassays), and the like. Amounts of purified polypeptide in solution may also be determined by physical methods, e.g. photometry. Methods of quantifying a particular polypeptide in a mixture usually rely on specific binding, e.g., of antibodies. Specific detection and quantitation methods exploiting the specificity of antibodies comprise for example immunoassay methods. For example, concentration/amount of marker/indicator proteins of the present invention in a patient sample may be determined by enzyme linked-immunosorbent assay (ELISA). Alternatively, Western Blot analysis or immunostaining can be performed. Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

As mentioned above, the expression level of the marker/indicator proteins according to the present invention may also be reflected in an increased expression of the corresponding gene(s) encoding the VEGFA, VEGFR2 and/or PLGF. Therefore, a quantitative assessment of the gene product prior to translation (e.g. spliced, unspliced or partially spliced mRNA) can be performed in order to evaluate the expression of the corresponding gene(s). The person skilled in the art is aware of standard methods to be used in this context or may deduce these methods from standard textbooks (e.g. Sambrook, 2001, loc. cit.). For example, quantitative data on the respective concentration/amounts of mRNA encoding one or more of VEGFA, VEGFR2 and/or PLGF can be obtained by Northern Blot, Real Time PCR and the like.

In a further aspect of the invention, the kit of the invention may advantageously be used for carrying out a method of the invention and could be, inter alia, employed in a variety of applications, e.g., in the diagnostic field or as a research tool. The parts of the kit of the invention can be packaged individually in vials or in combination in containers or multicontainer units. Manufacture of the kit follows preferably standard procedures which are known to the person skilled in the art. The kit or diagnostic compositions may be used for detection of the expression level of one or more of VEGFA, VEGFR2 and PLGF in accordance with the herein-described methods of the invention, employing, for example, immunohistochemical techniques described herein.

Although exemplified by the use of bevacizumab, the invention encompasses the use of other angiogenesis inhibitors as known in the art for use in combination with standard chemotherapy regimens. The terms “angiogenesis inhibitor” as used herein refers to all agents that alter angiogenesis (e.g. the process of forming blood vessels) and includes agents that block the formation of and/or halt or slow the growth of blood vessels. Nonlimiting examples of angiogenesis inhibitors include, in addition to bevacizumab, pegaptanib, sunitinib, sorafenib and vatalanib. Preferably, the angiogenesis inhibitor for use in accordance with the methods of the present invention is bevacizumab. As used herein, the term “bevacizumab” encompass all corresponding anti-VEGF antibodies or anti-VEGF antibody fragments, that fulfil the requirements necessary for obtaining a marketing authorization as an identical or biosimilar product in a country or territory selected from the group of countries consisting of the USA, Europe and Japan.

For use in the detection methods described herein, the skilled person has the ability to label the polypeptides, for example antibodies, or oligonucleotides encompassed by the present invention. As routinely practiced in the art, hybridization probes for use in detecting mRNA levels and/or antibodies or antibody fragments for use in immunoassay methods can be labelled and visualized according to standard methods known in the art, nonlimiting examples of commonly used systems include the use of radiolabels, enzyme labels, fluorescent tags, biotin-avidin complexes, chemiluminescence, and the like.

The person skilled in the art, for example the attending physician, is readily in a position to administer the bevacizumab in combination with a chemotherapy regimen to the patient/patient group as selected and defined herein. In certain contexts, the attending physician may modify, change or amend the administration schemes for the bevacizumab and the chemotherapy regimen in accordance with his/her professional experience. Therefore, in certain aspects of the present invention, a method is provided for the treatment or improving the overall survival and/or progression-free survival of a patient suffering from or suspected to suffer from pancreatic cancer, in particular metastatic pancreatic cancer, with bevacizumab in combination with a chemotherapy regimen, whereby said patient/patient group is characterized in the assessment of a biological sample from the patient (in particular a blood plasma sample), said sample exhibiting an increased expression level of one or more of VEGFA, VEGFR2 and PLGF relative to control levels established in patients diagnosed with pancreatic cancer, in particular metastatic pancreatic cancer. The present invention also provides for the use of bevacizumab in the preparation of pharmaceutical composition for the treatment of a patient suffering from or suspected to suffer from pancreatic cancer, in particular metastatic pancreatic cancer, wherein the patients are selected or characterized by the herein disclosed protein marker/indicator status (i.e., one or more of an increased expression level of VEGFA, VEGFR2 and PLGF relative to control levels established in patients diagnosed with pancreatic cancer, in particular metastatic pancreatic cancer.

The figures show:

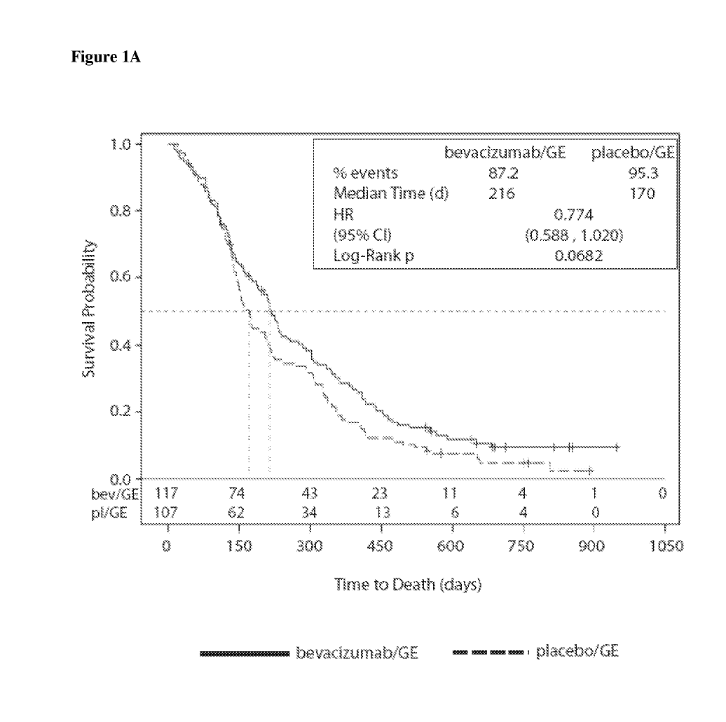

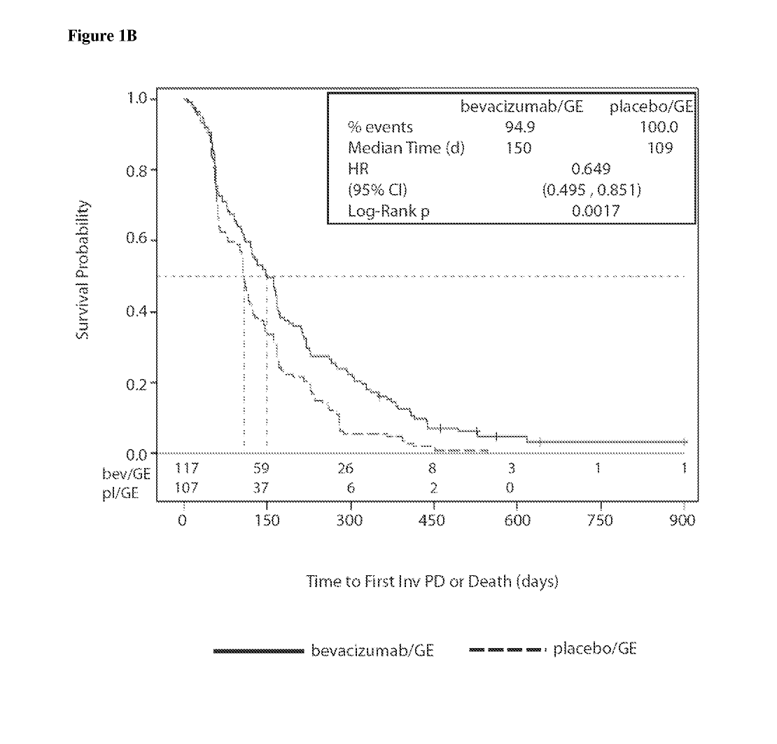

FIG. 1: Kaplan Meier Curves for Overall Survival (FIG. 1A) and for Progression Free Survival (FIG. 1B) for bevacizumab plus gemcitabine-erlotinib therapy versus control placebo plus gemcitabine-erlotinib therapy for patients being treated for metastatic pancreatic cancer. In the figures, the solid line represents bevacizumab/gemcitabine-erlotinib treatment and the dashed line represents placebo/gemcitabine-erlotinib treatment.

FIG. 2: Kaplan Meier Curves for association with treatment effect on Overall Survival for the marker VEGFA (FIG. 2A) and for association with treatment effect on Progression free survival for the marker VEGFA (FIG. 2B), for both high (≧152.9 pg/ml) and low (<152.9 pg/ml) expression levels, for bevacizumab plus gemcitabine-erlotinib therapy versus control placebo plus gemcitabine-erlotinib therapy for patients being treated for metastatic pancreatic cancer. In the figures, the solid line represents bevacizumab/gemcitabine-erlotinib treatment and the dashed line represents placebo/gemcitabine-erlotinib treatment.

FIG. 3: Kaplan Meier Curves for association with treatment effect on Overall Survival for the marker VEGFR2, for both high (≧9.9 ng/ml) and low (<9.9 ng/ml) expression levels, for bevacizumab plus gemcitabine-erlotinib therapy versus control placebo plus gemcitabine-erlotinib therapy for patients being treated for metastatic pancreatic cancer. In the figures, the solid line represents bevacizumab/gemcitabine-erlotinib treatment and the dashed line represents placebo/gemcitabine-erlotinib treatment.

FIG. 4: Kaplan Meier Curves for association with treatment effect on Progression Free Survival for the marker PLGF, for both optimized high (≧36.5 pg/ml) and low (<36.5 pg/ml) expression levels, for bevacizumab plus gemcitabine-erlotinib therapy versus control placebo plus gemcitabine-erlotinib therapy for patients being treated for metastatic pancreatic cancer. In the figures, the solid line represents bevacizumab/gemcitabine-erlotinib treatment and the dashed line represents placebo/gemcitabine-erlotinib treatment.

FIG. 5: Kaplan Meier Curves for association with treatment effect on Overall Survival for the markers VEGFA and VEGFR2 (FIG. 5A), as a combined expression level for both high (Formula 1≧−0.1) and low (Formula 1<−0.1) expression levels, and VEGFA and PLGF (FIG. 5B), as a combined expression level for both high (Formula 2≧−0.042) and low (Formula 2<−0.042) expression levels, for bevacizumab plus gemcitabine-erlotinib therapy versus control placebo plus gemcitabine-erlotinib therapy for patients being treated for metastatic pancreatic cancer. In the figures, the solid line represents bevacizumab/gemcitabine-erlotinib treatment and the dashed line represents placebo/gemcitabine-erlotinib treatment.

FIG. 6: Kaplan Meier Curves for association with treatment effect on Progression Free Survival for the markers VEGFA and VEGFR2 (FIG. 6A), as a combined expression level for both high (Formula 1≧−0.1) and low (Formula 1<−0.1) expression levels, and VEGFA and PLGF (FIG. 6B), as a combined expression level for both high (Formula 2≧−0.042) and low (Formula 2<−0.042) expression levels, for bevacizumab plus gemcitabine-erlotinib therapy versus control placebo plus gemcitabine-erlotinib therapy for patients being treated for metastatic pancreatic cancer. In the figures, the solid line represents bevacizumab/gemcitabine-erlotinib treatment and the dashed line represents placebo/gemcitabine-erlotinib treatment.

FIG. 7: Kaplan Meier Curve for association with treatment effect on Overall Survival for the markers for the markers VEGFA, VEGFR2 and PLGF (FIG. 7A), as a combined expression level for both high (Formula 3≧0.837) and low (Formula 3<0.837) expression levels, and for association with treatment effect on Progression Free Survival for the makers VEGFA, VEGFR2 and PLGF (FIG. 7B), as a combined expression level for both high (Formula 3≧0.837) and low (Formula 3<0.837) expression levels, for bevacizumab plus gemcitabine-erlotinib therapy versus control placebo plus gemcitabine-erlotinib therapy for patients being treated for metastatic pancreatic cancer. In the figure, the solid line represents bevacizumab/gemcitabine-erlotinib treatment and the dashed line represents placebo/gemcitabine-erlotinib treatment.

FIG. 8: SEQ ID NO:1, Exemplary amino acid sequence of VEGFA.

FIG. 9: SEQ ID NO:2, Exemplary amino acid sequence of VEGFR2.

FIG. 10: SEQ ID NO:3, Exemplary amino acid sequence of PLGF.

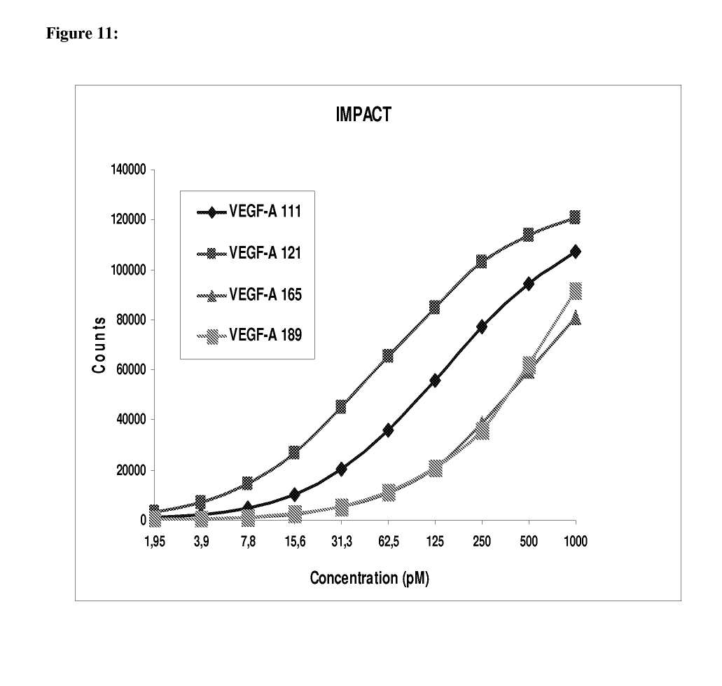

FIG. 11: Measurements of increasing concentrations of VEGF₁₁₁, VEGF₁₂₁, VEGF₁₆₅ and VEGF₁₈₉ as measured on an IMPACT chip.

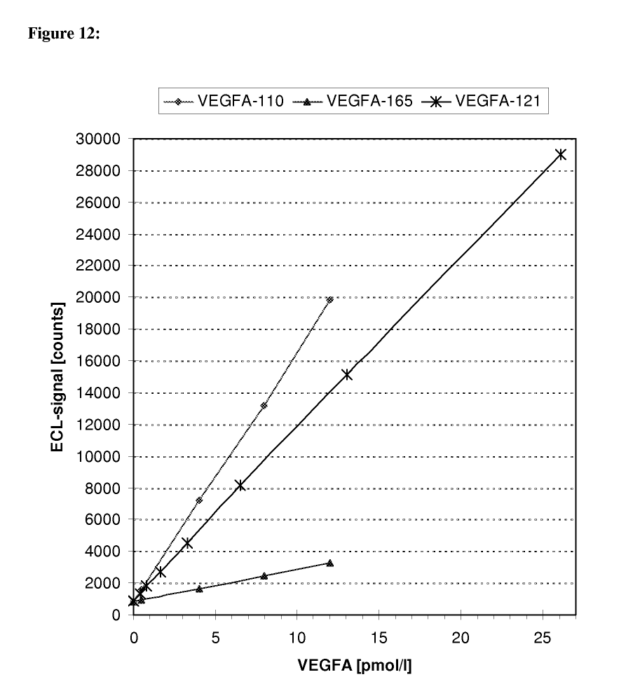

FIG. 12: Measurements of increasing concentrations of VEGF₁₁₀, VEGF₁₂₁, and VEGF₁₆₅ as measured using the Elecsys® Assay on the automated Elecsys® analyzer.

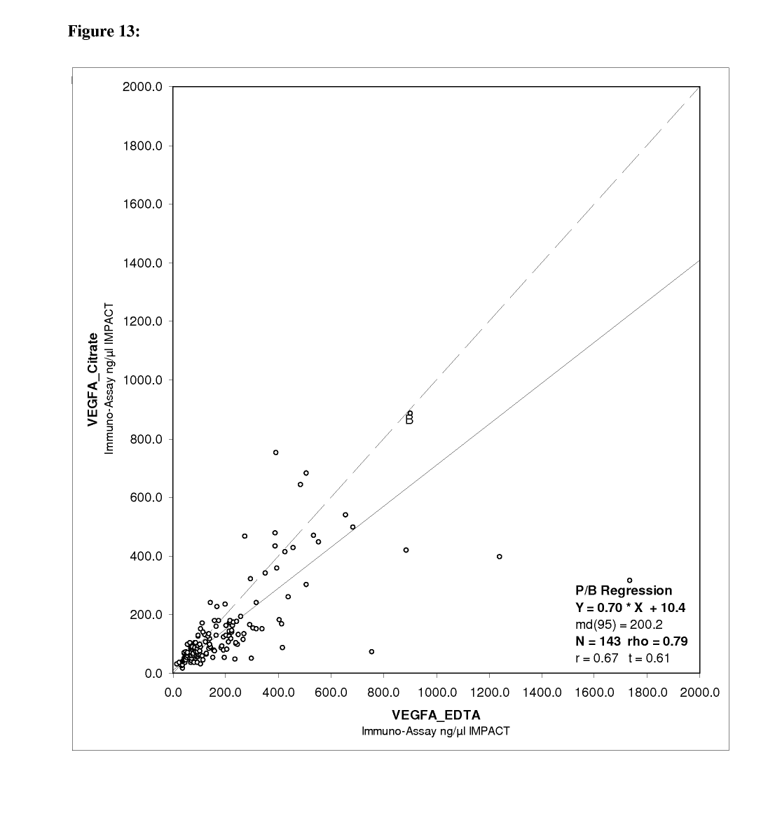

FIG. 13: Data from EDTA- and Citrate samples from the same patients measured twice with the IMPACT assay. The VEGFA concentration is about 40% higher for EDTA-plasma than for Citrate with a Spearman correlation for the EDTA-Citrate method comparison of about 0.8.

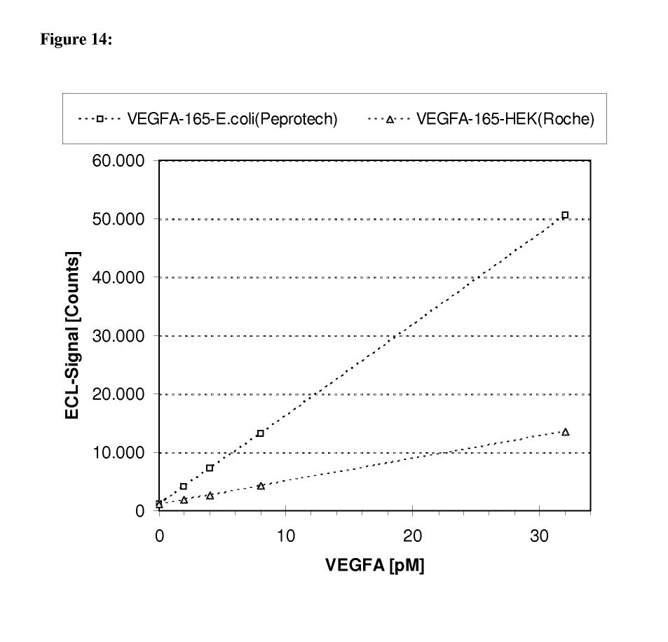

FIG. 14: Shown are the counts (ECL-signal) measured when increasing concentrations of VEGF₁₆₅, produced recombinantly in E. coli or in HEK-cells, respectively, were measured on the automated Elecsys® analyzer.

The present invention is further illustrated by the following non-limiting illustrative example.

### EXAMPLE 1

Patients with metastatic pancreatic adenocarcinoma were randomized to gemicitamibe-erlotinib plus bevacizumab (n=306) or placebo (n=301).

Blood plasma samples were collected from patients participating in a randomized phase-III study comparing the results of adding bevacizumab to gemicitamibe-erlotinib therapy for the treatment of metastatic pancreatic cancer (the B017706 study, see FIG. 1, also see Van Cutsem, J. Clin. Oncol. 2009 27:2231-2237). Patients with metastatic pancreatic adenocarcinoma were randomized to gemicitamibe-erlotinib plus bevacizumab (n=306) or placebo (n=301). Patients with metastatic pancreatic adenocarcinoma were randomly assigned to receive gemcitabine (1,000 mg/m²/week), erlotinib (100 mg/day), and bevacizumab (5 mg/kg every 2 weeks) or gemcitabine, erlotinib, and placebo.

An investigation of the status of biomarkers related to angiogenesis and tumorigenesis revealed that the expression levels of three biomarkers relative to control levels determined in the entire biomarker patient population correlated with an improved treatment parameter. In particular, patients exhibiting a higher expression level of VEGFA relative to control levels determined in the entire biomarker patient population, demonstrated a prolonged overall survival and a prolonged progression free survival in response to the addition of bevacizumab to gemicitamibe-erlotinib therapy. Patients exhibiting a higher expression level of VEGFR2 relative to control levels determined in the entire biomarker patient population, demonstrated a prolonged overall survival in response to the addition of bevacizumab to gemicitamibe-erlotinib therapy. Patient exhibiting a higher expression level of PLGF relative to control levels determined in the entire biomarker patient population, demonstrated a prolonged progression free survival in response to the addition of bevacizumab to gemicitamibe-erlotinib therapy. Also patients exhibiting higher combined expression level of VEGFA and VEGFR2 relative to control levels determined in the entire biomarker patient population, demonstrated a prolonged overall survival and a prolonged progression free survival in response to the addition of bevacizumab to gemicitamibe-erlotinib therapy. In addition, patients exhibiting higher combined expression level of VEGFA and PLGF relative to control levels determined in the entire patient population, demonstrated a prolonged overall survival and a prolonged progression free survival in response to the addition of bevacizumab to gemcitamibe-erlotinib therapy. Patients exhibiting higher combined expression level of VEGFA, VEGFR2 and PLGF relative to control levels determined in the entire patient population, demonstrated a prolonged overall survival and a prolonged progression free survival in response to the addition of bevacizumab to gemcitamibe-erlotinib therapy

### Patients and Immunochemical Methods

A total of 607 patients participated in the BO17706 study, and blood plasma samples from 224 of the participants were available for biomarker analysis. The baseline characteristics of the 224 patients in the biomarker analysis are provided in Table 1.

TABLE 1
Baseline characteristics: biomarker population (n = 224)

| | |
|---|---|
| bevacizumab | placebo |

| | N | (%) | N | (%) |
|---|---|---|---|---|
| Sex | | | | |
| Female | 45 | 38.46 | 32 | 29.91 |
| Male | 72 | 61.54 | 75 | 70.09 |
| Age Category (years) | | | | |
| <65 | 73 | 62.39 | 71 | 66.36 |
| >=65 | 44 | 37.61 | 36 | 33.64 |
| KPS (%) Category at Baseline | | | | |
| <80% | 15 | 12.82 | 13 | 12.15 |
| >=80% | 102 | 87.18 | 94 | 87.85 |
| VAS Category at Baseline | | | | |
| below baseline (not available) | 10 | 8.55 | 16 | 14.95 |
| <20 | 68 | 58.12 | 56 | 52.34 |
| >=20 | 39 | 33.33 | 35 | 32.71 |
| CRP Category (median value) | | | | |
| at Baseline (mg/dL) | | | | |
| below baseline (not available) | 13 | 11.11 | 9 | 8.41 |
| <=1.4 | 52 | 44.44 | 49 | 45.79 |
| >1.4 | 52 | 44.44 | 49 | 45.79 |

VAS: Visual Analogue Scale of Pain
KPS: Karnofsky Performance Score

### Blood Plasma Analysis

Plasma samples were collected after randomization and before any study treatment was given to the patients and VEGFA, vascular endothelial growth factor receptor 1 (VEGFR1), VEGFR2, PLGF and E-SELECTIN were measured using a multiplex ELISA assay (Impact) from Roche Diagnostics GmbH.

### IMPACT Multiplex Assay Technology

Roche Professional Diagnostics (Roche Diagnostics GmbH) has developed a multimarker platform under the working name IMPACT (Immunological MultiParameter Chip Technology). The technology is based on a small polystyrene chip manufactured by procedures as disclosed in EP 0939319 and EP 1610129. The chip surface was coated with a streptavidin layer, onto which the biotinylated antibodies were then spotted for every assay. For each marker, spots of antibodies were loaded in a vertical line onto the chip. During the assay, the array was probed with specimen samples containing the specific analytes.

The plasma volume required per specimen for measuring all markers on one chip was 8 μL, which was applied together with 32 μL of an incubation buffer (50 mM HEPES pH 7.2, 150 mM NaCl, 0.1% Thesit, 0.5% bovine serum albumin and 0.1% Oxypyrion as a preservative agent). After incubation for 12 minutes and washing of the chip using a washing buffer (5 mM Tris pH 7.9, 0.01% Thesit and 0.001% Oxypyrion) the digoxigenylated monoclonal antibody mix was added (40 μL of incubation buffer including a mix of the analyte-specific antibodies labeled with Digoxigenin) and was incubated for an additional 6 minutes to bind onto the captured analytes. The second antibody was finally detected with 40 μL of a reagent buffer (62.5 mM TAPS pH 8.7, 1.25 M NaCl, 0.5% bovine serum albumin, 0.063% Tween 20 and 0.1% Oxypyrion) including an anti-digoxigenin antibody conjugate coupled with fluorescent latex. Using this label, 10 individual binding events in a single spot could be detected, resulting in very high sensitivity down to the fmol/L concentration. Chips were transported into the detection unit, and a charge coupled device (CCD) camera generated an image that was transformed into signal intensities using dedicated software. Individual spots were automatically located at predefined positions and quantified by image analysis. For each marker, lines of 10-12 spots were loaded on the chips, and a minimum of 5 spots was required to determine the mean concentration of samples. The advantages of the technology are the ability of multiplexing up to 10 parameters in a sandwich or competitive format. The calibrators and patient samples were measured in duplicate. One run was designed to contain a total of 100 determinations, including 2 multi-controls as a run control. Since some of the selected analytes react with each other (i.e. VEGFA and PLGF with VEGFR1 or VEGRF2 or VEGFA forms heterodimers with PLGF), the 5 analytes were divided on three different chips as follows:

### Chip 1: VEGFA

### Chip 2: VEGFR1, VEGFR2, E-Selectin

### Chip 3: PLGF

The following antibodies were used for the different assays:

| | Capture | Manu- | Detection | Manu- |
|---|---|---|---|---|
| Analyte | antibody | facturer | antibody | facturer |
| VEGFA | <VEGF- | Bender | <VEGF>M- | R&D |
| | A>M-3C5 | RELIATech | 26503 | Systems |
| VEGFR1 | <VEGF- | Roche | <VEGF- | Roche |
| | R1>M-49560 | Diagnostics | R1>M-49543 | Diagnostics |
| VEGFR2 | <VEGF- | R&D | <VEGF- | R&D |
| | R2>M-89115 | Systems | R2>M-89109 | Systems |
| E-Selectin | <E- | R&D | <E- | R&D |
| | Selectin>M- | Systems | Selectin>M- | Systems |
| | BBIG-E5 | | 5D11 | |
| PLGF | <PLGF>M- | Roche | <PLGF>M- | Roche |
| | 2D6D5 | Diagnostics | 6A11D2 | Diagnostics |

### Statistical Analysis

Sample median was used to dichotomize biomarker values as low (below median) or high (above median).

Hazard Ratio of treatment effect in sub-group of patients with high or low biomarker levels were estimated with proportional hazard cox regression analysis.

In addition, proportional hazard cox regressions was used to evaluate the association between biomarker level and treatment effect. The model included the following covariates: trial treatment, biomarker level, interaction term of treatment by biomarker level. Wald test for the interaction term was used to determined the association between biomarker level and treatment effect. P-value below 0.05 was considered significant.

### Results

### Blood Plasma Markers

The baseline descriptive statistics of the biomarkers are presented in Table 2.

TABLE 2
Descriptive Statistics of Biomarker Values (Baseline)

| | VEGFA | VEGFR2 | PlGF |
|---|---|---|---|
| | (pg/mL) at | (ng/mL) at | (pg/mL) at |
| | baseline | baseline | baseline |
| min | 3.06 | 0.23 | 0 |
| qu 25% | 80.08 | 7.9 | 32.9 |
| median | 152.80 | 9.9 | 37.8 |
| qu 75% | 275.90 | 12.6 | 43.6 |
| max | 2127.00 | 58.1 | 142.3 |
| mean | 215.30 | 10.4 | 39.4 |
| sd | 254.8 | 4.7 | 12.5 |

Table 3 presents the univariate analysis of the association of the selected biomarkers with treatment effect on overall survival.

TABLE 3
Association with treatment effect on Overall
Survival - (uni-variate analysis)

| | | P-value for |
|---|---|---|
| | HR (95% CI) | interaction |
| VEGFA low | 1.018 (0.69, 1.5) | 0.0308 |
| VEGFA high | 0.558 (0.37, 0.83) | |
| VEGFR2 low | 1.057 (0.72, 1.55) | 0.0461 |
| VEGFR2 high | 0.583 (0.39, 0.87) | |
| PLGF low | 1.048 (0.67, 1.63) | 0.089 |
| PLGF high | 0.659 (0.46, 0.95) | |

In this analysis, for VEGFA, Low VEGFA<152.9 pg/ml and High VEGFA≧152.9 pg/ml, for VEGFR2, Low VEGFR2<9.9 ng/ml and High VEGFRA≧9.9 ng/ml, and for PLGF, Low PLGF<36.5 pg/ml and High PLGF≧36.5 pg/ml, were used.

For VEGFA and VEGFR2 the cut-off levels were determined as sample data median value, such that 50% of patients have high expression and 50% of patients have low expression, as per pre-determined analysis plan. The PLGF cut-off levels were determined as 42^{nd} percentile of the data. Accordingly, 58% of patients have high expression of PLGF and 42% have low expression. The cut-off was determined in order to increase the statistical difference between treatment effect in high and low level subgroup.

This result table shows that the Hazard Ratio for treatment effect is significantly better in the subset of patients with high VEGFA compared to patients with low VEGFA. This result table also shows that the Hazard Ratio for treatment effect is significantly better in the subset of patients with high VEGFR2 compared to patients with low VEGFR2. Therefore, VEGFA and VEGFR2 are each independent predictive biomarkers for Bevacizumab treatment effect on overall survival.

Table 4 presents the univariate analysis of the association of the selected biomarkers with treatment effect on progression free survival.

TABLE 4
Association with treatment effect on Progression
Free Survival (univariate analysis)

| | | P-value for |
|---|---|---|
| | HR (95% CI) | interaction |
| VEGFA low | 0.771 (0.53, 1.13) | 0.0603 |
| VEGFA high | 0.522 (0.35, 0.78) | |
| VEGFR2 low | 0.773 (0.53, 1.12) | 0.4012 |
| VEGFR2 high | 0.541 (0.36, 0.81) | |
| PLGF low | 0.957 (0.63, 1.46) | 0.0136 |
| PLGF high | 0.505 (0.35, 0.73) | |

In this analysis, for VEGFA, Low VEGFA<152.9 pg/ml and High VEGFA≧152.9 pg/ml, for VEGFR2, Low VEGFR2<9.9 ng/ml and High VEGFRA≧9.9 ng/ml, and for PLGF, Low PLGF<36.5 pg/ml and High PLGF≧36.5 pg/ml, were used. For VEGFA and VEGFR2 the cut-off levels were determined as sample data median value, such that 50% of patients have high expression and 50% of patients have low expression, as per pre-determined analysis plan. The PLGF cut-off levels were determined as 42^{nd} percentile of the data. Accordingly, 58% of patients have high expression of PLGF and 42% have low expression. The cut-off was determined in order to increase the statistical difference between treatment effect in high and low level subgroup.

This result table shows that the Hazard Ratio for treatment effect is significantly better in the subset of patients with high VEGFA compared to patients with low VEGFA. This result table also shows that the Hazard Ratio for treatment effect is significantly better in the subset of patients with high PLGF compared to patients with low PLGF. Therefore, VEGFA and PLGF are each independent predictive biomarkers for bevacizumab treatment effect on progression free survival.

Table 5 presents the analysis of biomarker combinations association with treatment effect on overall survival.

For this analysis the following equations were used:

norm(VEGFA)+1.3*norm(VEGFR2). Cut-point=median or 0 Formula 1

VEGFA+3.3*VEGFR2. Cut-point=median or 0 Equivalent formula

and

0.25*norm(VEGFA)+0.21*norm(PLGF), cut-point=median or 0 Formula 2

0.19*VEGFA+0.67*PLGF, cut-point=median or 4.8 Equivalent formula

Where we use log 2 transformation and

$${x}_{i} \to norm ( {x}_{i} ) = \frac{log 2 ( {x}_{i} ) - median ( log 2 ( x ) )}{mad ( log 2 ( x ) )}$$

TABLE 5
Association with treatment effect on
Overall Survival (bi-marker analysis)

| | | P-value for |
|---|---|---|
| | HR (95% CI) | interaction |
| VEGFA & VEGFR2 low | 1.317 (0.89, 1.94) | 0.0002 |
| VEGFA & VEGFR2 high | 0.42 (0.28, 0.64) | |
| VEGFA & PLGF low | 1.101 (0.74, 1.64) | 0.0096 |
| VEGFA & PLGF high | 0.546 (0.37, 0.81) | |

In this analysis, a high combined expression level of VEGFA and VEGFR2 is (Formula 1≧−0.10) and a low combined expression of VEGFA and VEGFR2 is (Formula 1<−0.10), and a high combined expression level of VEGFA and PLGF is (Formula 2≧−0.042) and a low combined expression of VEGFA and PLGF is (Formula 2<−0.042).

This results table shows that the Hazard Ratio for treatment effect is significantly better in the subset of patients with high VEGFA & VEGFR2 combination compared to patients with low VEGFA & VEGFR2 combination. This result table also shows that the Hazard Ratio for treatment effect is significantly better in the subset of patients with high VEGA & PLGF combination compared to patients with low VEGFA & PLGF combination. Therefore, VEGFA & VEGFR2 combination and VEGFA & PLGF combination are each independent predictive biomarkers for bevacizumab treatment effect on overall survival.

Table 6 presents the analysis of biomarker combinations association with treatment effect on progression free survival.

For this analysis the following equations were used:

norm(VEGFA)+1.3*norm(VEGFR2). Cut-point=median or 0 Formula 1

VEGFA+3.3*VEGFR2. Cut-point=median or 0 Equivalent formula

and

0.25*norm(VEGFA)+0.21*norm(PLGF), cut-point=median or 0 Formula 2

0.19*VEGFA+0.67*PLGF, cut-point=median or 4.8 Equivalent formula

Where we use log 2 transformation and

$${x}_{i} \to norm ( {x}_{i} ) = \frac{log 2 ( {x}_{i} ) - median ( log 2 ( x ) )}{mad ( log 2 ( x ) )}$$

TABLE 6
Association with treatment effect on Progression
Free Survival (bi-marker analysis)

| | | P-value for |
|---|---|---|
| | HR (95% CI) | interaction |
| VEGFA & VEGFR2 low | 0.984 (0.68, 1.43) | 0.0040 |
| VEGFA & VEGFR2 high | 0.411 (0.26, 0.64) | |
| VEGFA & PLGF low | 0.936 (0.64, 1.37) | 0.0011 |
| VEGFA & PLGF high | 0.426 (0.28, 0.64) | |

In this analysis, a high combined expression level of VEGFA and VEGFR2 is (Formula 1≧−0.10) and a low combined expression of VEGFA and VEGFR2 is (Formula 1<−0.10), and a high combined expression level of VEGFA and PLGF is (Formula 2≧−0.042) and a low combined expression of VEGFA and PLGF is (Formula 2<−0.042).

This results table shows that the Hazard Ratio for treatment effect is significantly better in the subset of patients with high VEGFA & VEGFR2 combination compared to patients with low VEGFA & VEGFR2 combination. This result table also shows that the Hazard Ratio for treatment effect is significantly better in the subset of patients with high VEGA & PLGF combination compared to patients with low VEGFA & PLGF combination. Therefore, VEGFA & VEGFR2 combination and VEGFA & PLGF combination are each independent predictive biomarkers for bevacizumab treatment effect on progression free survival.

Tables 7 and Table 8 present the analysis of biomarker combinations of VEGFA, VEGFR2 and PLGF association with treatment effect on overall survival and progression free survival, respectively.

In this analysis, the following equation was used:

0.0127*ln(PLGF+1)+0.144*ln(VEGFR2+1)+0.0949*ln(VEGFA+1) Formula 3

Where ln=log basis e

TABLE 7
Association with treatment effect on Overall
Survival (tri-marker analysis)

| | | P-value for |
|---|---|---|
| Overall Survival | HR (95% CI) | interaction |
| VEGFA & VEGFR2 & PLGF low | 1.051 (0.71, 1.55) | 0.0033 |
| VEGFA & VEGFR2 & PLGF high | 0.554 (0.38, 0.8) | |

TABLE 8
Association with treatment effect on Progression
Free Survival (tri-marker analysis)

| | | P-value for |
|---|---|---|
| Progression Free Survival | HR (95% CI) | interaction |
| VEGFA & VEGFR2 & PLGF low | 0.974 (0.64, 1.48) | 0.0096 |
| VEGFA & VEGFR2 & PLGF high | 0.488 (0.34, 0.71) | |

In this analysis, for overall survival, a high combined expression level of VEGFA, VEGFR2 and PLGF is (Formula 3≧0.837) and a low combined expression of VEGFA, VEGFR2 and PLGF is (Formula 3<0.837), and for progression free survival, a high combined expression level of VEGFA, VEGFR2 and PLGF is (Formula 3≧0.837) and a low combined expression of VEGFA, VEGFR2 and PLGF is (Formula 3<0.837).

This results table shows that the Hazard Ratio for treatment effect is significantly better in the subset of patients with high VEGFA & VEGFR2 & PLGF combination compared to patients with low VEGFA & VEGFR2 & PLGF combination. Therefore, VEGFA & VEGFR2 & PLGF combination is a predictive biomarkers for bevacizumab treatment effect on progression free survival.

This results table also shows that for overall survival the Hazard Ratio for treatment effect is significantly better in the subset of patients with high VEGFA & VEGFR2 & PLGF combination compared to patients with low VEGFA & VEGFR2 & PLGF combination. Therefore, VEGFA & VEGFR2 & PLGF combination is a predictive biomarkers for Bevacizumab treatment effect on overall survival.

### EXAMPLE 2

### Detection of Shorter Isoforms of VEGF-A Using the Impact Assay

This example demonstrates that, based on the antibodies used for detection of VEGF-A on the IMPACT platform, the shorter isoforms of VEGF-A are preferentially measured as compared to the longer isoforms of VEGF-A.

The assay was performed as described above under the section relating to the IMPACT technology using the antibodies listed in the table before the “statistical analysis” section.

Four different VEGF-A forms, i.e. VEGF₁₁₁, VEGF₁₂₁, VEGF₁₆₅ and VEGF₁₈₉ were available and used in the analysis. VEGF₁₁₁, VEGF₁₂₁ (both derived from expression in E. coli), and VEGF₁₆₅ (obtained recombinantly in an insect cell line) was purchased from R&D Systems, Minneapolis, USA and VEGF₁₈₉ was obtained from RELIATech, Wolfenbüttel, Germany. It has turned out later that VEGF189 appears to be rather unstable and that the data obtained with that material cannot be relied upon. As shown in FIG. 11 the shorter isoforms having 111 or 121 amino acids, respectively, which had been produced in E. coli and are not secondarily modified, e.g., not glycosylated, are detected better as compared to the longer isoforms with 165 amino acids. VEGF165 had been obtained in an insect cell line and is at least partially glycosylated. The biologically interesting plasmin cleavage product VEGF₁₁₀ was not available for testing at this point in time, but is has to be expected that detection of this isoform will be comparable to what is seen for the VEGF-molecule with 111 amino acids.

### EXAMPLE 3

### Detection of Short VEGF Isoforms Using the Elecsys® Analyzer

This example describes experiments demonstrating that an assay using the Elecsys® Analyzer and a corresponding assay can be used to detect short VEGF isoforms in human plasma.

The VEGF-A assay was transferred from IMPACT to the automated in-vitro diagnostics system Elecsys® (Roche Diagnostics GmbH, Mannheim). The same capture antibody as in the IMPACT Assay, <hVEGF-A>-m3C5 (RELIATech, Wolfenbüttel) was used, while the capture antibody <hVEGF-A>-m25603 (R&D Systems, Minneapolis) used on the IMPACT system was replaced by <hVEGF-A>-mA4.6.1 (Genentech, South San Francisco).

The immunoassays running on the automated Elecsys® system are immuno assays using electrochemiluminescense (ECLIA) as the signal generating technology. In the present sandwich assay the biotinylated capture antibody binds to streptavidin coated, magnetic microparticles and the ruthenylated detection antibody allows for signal generation. 75 μl of biotinylated <VEGF-A>-m3C5 at 1.5 μg/ml and 75 μl of ruthenylated <VEGF-A>M-A.4.6.1 at 2 μg/ml both in reaction buffer (50 mM Tris (pH 7.4), 2 mM EDTA, 0.1% thesit, 0.2% bovine IgG, 1.0% bovine serum albumin) were incubated for 9 minutes with 20 μl of sample. 30 μl of a microparticle suspension was added after the first 9 minutes of incubation and the whole mixture then incubated for an additional 9 minutes. During these incubation steps an antibody analyte antibody sandwich is formed that is bound to the microparticles. Finally the microparticles were transferred to the detection chamber of the Elecsys system for signal generation and readout.

The cleavage product/isoform preference of the Elecsys® VEGF-A assay was assessed with purified recombinant proteins: VEGF 110 (produced by plasmin cleavage at Genentech, South San Francisco), VEGF 121 and VEGF 165 (both produced in an insect cell line and supplied by R&D Systems, Minneapolis). The preferential binding of short VEGF isoforms that had been seen with the IMPACT® Assay was confirmed in the Elecsys assay. As shown in FIG. 12, in the Elecsys® assay the isoforms VEGF 121 and the plasmin cleavage product VEGF 110, respectively, both were detected with an approximately 5-fold higher sensitivity than VEGF 165.

### EXAMPLE 4

### Detection of Short VEGF Isoforms in Plasma Collected in Na Citrate and EDTA

Paired plasma samples were collected from patients with HER2+ locally recurrent or metastatic breast cancer in both an EDTA monovette (5 mL)- and Citrate Monovette collection tube (5 mL). Within 30 minutes of blood collection, blood tubes were placed into the centrifuge and spun 1500 g at room temperature for 10 minutes, until cells and plasma were separated. Immediately after centrifugation, the plasma was carefully transferred into a propylene transfer tube and then aliquotted equally into 2 storage tubes (half volume each approximately 1.25 mL) using a pipette. The levels of VEGF-A in the samples were measured using the IMPACT Assay described above. As shown in FIG. 13, the VEGFA concentration is about 40% higher for plasma samples collected and stored in EDTA compared to plasma samples collected and stored in citrate with a Spearman correlation for the EDTA-Citrate MC of about 0.8 for baseline samples collected prior to treatment.

### EXAMPLE 5

### Comparative Measurement of Unmodified and Modified VEGF165 on the Elecsys Analyzer

This example describes experiments demonstrating that the Elecsys® Analyzer and a corresponding assay can be used to detect unmodified VEGF in human plasma.

The VEGF-A assay was transferred from IMPACT to the automated in-vitro diagnostics system Elecsys® (Roche Diagnostics GmbH, Mannheim). The same capture antibody as in the IMPACT assay, <hVEGF-A>-m3C5 (RELIATech GmbH, Wolfenbüttel) was used, while the detection antibody <hVEGF-A>-m25603 (R&D Systems, Minneapolis) used on the IMPACT system was replaced by <hVEGF-A>-mA4.6.1 (Genentech, South San Francisco).

The immunoassays running on the automated Elecsys system are immuno assays using electrochemiluminescense (ECLIA) as the signal generating technology. In the present sandwich assay the biotinylated capture antibody binds to streptavidin coated, magnetic microparticles and the ruthenylated detection antibody allows for signal generation. 75 μl of biotinylated <VEGF-A>-m3C5 at 1.5 μg/ml and 75 μl of ruthenylated <VEGF-A>M-A.4.6.1 at 2 μg/ml both in reaction buffer (50 mM Tris (pH 7.4), 2 m M EDTA, 0.1% thesit, 0.2% bovine IgG, 1.0% bovine serum albumin) were incubated for 9 minutes with 20 μl of sample. 30 μl of a microparticle suspension was added after the first 9 minutes of incubation and the whole mixture then incubated for an additional 9 minutes. During these incubation steps an antibody-analyte-antibody sandwich is formed that is bound to the microparticles. Finally the microparticles were transferred to the detection chamber of the Elecsys system for signal generation and readout.

The preference of the Elecsys VEGF-A assay was assessed with purified recombinant proteins: VEGF165 (produced recombinantly in E. coli by Peprotech) and VEGF165 (produced recombinantly in HEK-cells at Roche Diagnostics, Germany). The preferential binding of unmodified VEGF165 that had been seen with the IMPACT assay was confirmed in the Elecsys assay. As shown in FIG. 14, in the Elecsys assay the unmodified VEGF 165 was detected with an approximately 5-fold higher sensitivity than modified VEGF 165.

1. A method for improving the treatment effect of a chemotherapy regimen of a patient suffering from pancreatic cancer by adding bevacizumab to said chemotherapy regimen, said method comprising:
(a) determining the protein expression level of one or more of VEGFA, VEGFR2 and PLGF in a patient sample; and
(b) administering bevacizumab in combination with a chemotherapy regimen to the patient having an increased expression level of one or more of VEGFA, VEGFR2 and PLGF relative to control expression levels determined in patients diagnosed with pancreatic cancer.

2. An in vitro method for the identification of a patient responsive to or sensitive to the addition of bevacizumab treatment to a chemotherapy regimen, said method comprising determining the protein expression level of one or more of VEGFA, VEGFR2 and PLGF in a sample from a patient suspected to suffer from or being prone to suffer from pancreatic cancer, whereby an increased expression level of one or more of VEGFA, VEGFR2 and PLGF relative to control expression levels determined in patients suffering from pancreatic cancer is indicative of a sensitivity of the patient to the addition of bevacizumab to said chemotherapy regimen.

3. An in vitro method of predicting the response to or sensitivity to the addition of bevacizumab to a chemotherapy regimen of a patient suspected to suffer from, suffering from or prone to suffer from pancreatic cancer comprising determining the protein expression level of one or more of VEGFA, VEGFR2 and PLGF in a patient sample.

4. The method of any one of claims 1 to 3, wherein the treatment effect is progression-free survival.

5. The method of any one of claims 1 to 3, wherein the treatment effect is overall survival.

6. The method of any one of claims 1 to 3, wherein the protein expression level determined is of VEGFA or PLGF.

7. The method of any one claims 1 to 3, wherein the protein expression level determined is of VEGFA or VEGFR2.

8. The method of any one of claims 1 to 3, wherein the protein expression level determined is a combined expression level of VEGFA and VEGFR2.

9. The method of any one of claims 1 to 3, wherein the protein expression level determined is a combined expression level of VEGFA and PLGF.

10. The method of any one of claims 1 to 3, wherein the protein expression level determined is a combined expression level of VEGFA, VEGFR2 and PLGF.

11. The method of any one of claims 1 to 3, wherein said expression level is detected by an immunoassay method.

12. The method of claim 11, wherein said immunoassay method is ELISA.

13. The method of any one of claims 1 to 3, wherein said patient sample is a blood sample.

14. The method of claim 13, wherein said patient sample is a blood plasma sample.

15. The method of any one of claims 1 to 3, wherein the pancreatic cancer is metastatic pancreatic cancer.

16. The method of claim 15, wherein said chemotherapy regimen comprises gemcitabine and erlotinib.

17. The method of any one of claims 1 to 3, wherein said patient is being co-treated with one or more anti-cancer therapies.

18. The method of claim 17, wherein said anti-cancer therapy is radiation.

19. The method of any one of claims 1 to 3, wherein said sample is obtained before neoadjuvant or adjuvant therapy.

20. The method of any one of claims 1 to 3, wherein said samples is obtained after neoadjuvant or adjuvant therapy.

21. A kit useful for identifying a patient suffering from, suspected to suffer from, or being prone to suffer from pancreatic cancer as being responsive to or sensitive to the addition of bevacizumab treatment to a chemotherapy regimen, the kit comprising polypeptides capable of determining the expression level of one or more of VEGFA, VEGFR2 and/or PLGF and instructions for use of the polypeptides to determine the level of VEGFA, VEGFR2 and/or PLGF in a sample from the patient, wherein an increase in the protein expression level of VEGFA, VEGFR2 and/or PLGF identifies a patient as being responsive to or sensitive to the addition of bevacizumab treatment to a chemotherapy regimen.

22. The kit of claim 21, wherein said polypeptide is suitable for use in an immunoassay method and/or is an antibody specific for VEGFA, VEGFR2 or PLGF.


SEQUENCE LISTING (3 sequences)

SEQ ID NO: 1 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 2 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 3 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR